(19) United States
(12) Patent Application Publication
(10) Pub. No.: US 2023/0330223 A1
(43) Pub. Date: Oct. 19, 2023

(54) TREATMENT OF HER2 NEGATIVE, MAMMAPRINT HIGH RISK 2 BREAST CANCER

(71) Applicant: Agendia N.V., Amsterdam (NL)

(72) Inventor: Annuska Maria GLAS, Amsterdam (NL)

(73) Assignee: Agendia N.V., Amsterdam (NL)

(21) Appl. No.: 17/921,543

(22) PCT Filed: Apr. 26, 2021
(86) PCT No.: PCT/NL2021/050275

§ 371 Date: Oct. 26, 2022

(30) Foreign Application Priority Data
Apr. 27, 2020 (EP) 20171467.2

(51) Int. Cl. A61K 39/395; C12Q 1/6886; C12Q 1/6874; A61K 31/337; A61K 31/502; A61K 31/55; A61K 31/454; A61K 31/5025; A61K 31/5517; A61P 35/00

(52) U.S. Cl. CPC A61K 39/3955; C12Q 1/6886; C12Q 1/6874; A61K 31/337; A61K 31/502; A61K 31/55; A61K 31/454; A61K 31/5025; A61K 31/5517; A61P 35/00; C12Q 2600/158; C12Q 2600/118; C12Q 1/6841

(57) ABSTRACT

The invention relates to a PARP inhibitor, an immune checkpoint inhibitor, or a combination thereof, for use as a medicament. The invention further relates to a kit of parts and to a pharmaceutical composition comprising a PARP inhibitor and an immune checkpoint inhibitor, preferably for use in a method of treating a breast cancer.

### CROSS-REFERENCE TO RELATED APPLICATIONS

This application is a U.S. National Phase application of International Application No. PCT/NL2021/050275, filed Apr. 26, 2021, which claims priority to European Patent application number EP 20171467.2, filed Apr. 27, 2020, the disclosures of which are incorporated herein by reference in their entireties.

### FIELD

The invention relates to methods for treatment of cancer, especially a breast cancer, using a combination of medicaments, thereby targeting multiple pathways.

### 1 INTRODUCTION

Cancer is a leading cause of death worldwide, accounting for an estimated total of 9.6 million deaths in 2018. The most common cancers are lung cancer, breast cancer and colorectal cancers, while lung cancer, colorectal cancer and stomach cancer are the most common causes of cancer death (The Global Cancer Observatory, 2019. Factsheet on cancers/39).

Early diagnosis and treatment of cancer may provide significant improvement to the lives of cancer patients. Some of the most common cancer types, such as breast cancer and colorectal cancer, have high cure rates when detected and treated at an early stage, before cancer cells have metastasized.

Although early stage breast cancer has a relative good prognosis, still approximately 30% of patients would develop a distant metastasis and die from the disease if not treated with adjuvant therapy after surgery (Rosen et al., 1989. J Clin Oncol 7: 355-366). It is common to treat hormone receptor positive breast cancer with adjuvant endocrine therapy. There are clinical factors such as size and grade and genomic signatures used by clinicians to identify patients who need adjuvant chemotherapy.

Breast cancer cells can be classified into molecular subtypes basal, luminal and HER2 positive, by simple hierarchical clustering of breast tumors according to their gene expression patterns (Perou et al., 2000. Nature 406: 747-7520. In general, these subtypes represent the Estrogen Receptor (ER), Progesterone Receptor (PR) and Human Epidermal growth factor Receptor 2 (HER2) status of the tumor: Basal-like breast cancers correlate best with triple negative (ER-negative, PR-negative, and HER2-negative tumors) breast cancers (Rakha et al., 2009. Clin Cancer Res 15: 2302-2310; Carey et al., 2007. Clin Cancer Res 13: 2329-2334). Luminal-like cancers are ER-positive (Nielsen et al., 2004. Clin Cancer Res 10: 5367-5374) and HER2 positive cancers have a high expression of the HER2 gene (Kauraniemi and Kallioniemi. 2006. Endocr Relat Cancer 13: 39-49). While this classification system has been developed without consideration of patient survival rates, the different molecular subtypes of breast cancer have different prognoses: luminal-like tumors have a more favorable outcome and basal-like and HER2 subgroups appear to be more sensitive to chemotherapy (Sorlie et al., 2001. Proc Natl Acad Sci USA 98: 10869-10874; Rouzier et al., 2005. Clin Cancer Res 11: 5678-5685; Liedtke et al., 2008. J Clin Oncol 26: 1275-1281; Krijgsman et al., 2012. Breast Cancer Res Treat 133: 37-47).

Patients with positive HER2 receptor usually receive anti HER2 therapy. Despite these treatment options there are still too many breast cancer patients that develop distant metastasis. New therapeutics are developed to target specific pathway defects in early stage breast cancer. Two classes that are tested are PARP inhibitors and PD-1/PDL-1 inhibitors. Some have shown promising effects in metastatic breast cancer. These classes are primarily tested in hormone receptor (ER and PR) negative breast cancer, such as BRCA-mutated breast cancers. These cancers often have a DNA repair deficient phenotype which might make them sensitive for PARP inhibitions. The genomic instability caused by DNA repair deficiency also causes these tumors to accumulate more mutations and therefor are easier to recognize by the patient's immune system which makes these tumors potentially sensitive for PD-1/PDL-1 inhibitors (Robson et al., 2017. N Engl J Med 377: 523-533; Schmid et al., 2018. N Engl J Med 379: 2108-2121).

Hormone receptor positive breast cancer does not often represent these phenotypes and PARP and PD-1 have shown little success in HR+ breast cancer.

The I-SPY 2 TRIAL (NCT01042379), sponsored by Quantum Leap Healthcare Collaborative, is a standing Phase 2 randomized, controlled, multi-center trial for women with newly diagnosed, locally advanced breast cancer (Stage II/III), and is designed to screen promising new treatments and identify which therapies are most effective in specific patient subgroups based on molecular characteristics (biomarker signatures). The trial is an adaptive study design assessing the combination of biologically targeted investigational drugs with standard chemotherapy in the neoadjuvant setting, compared to standard chemotherapy alone. The primary endpoint is to determine whether the combination of certain therapies increases the probability of pathological complete response (pCR) in the breast and the lymph nodes at the time of surgery (Barker et al., 2009. Clin Pharmacol Ther 86: 97-100).

Results from this program have shown that a combination of a PARP inhibitor (veliparib) and a platinum-based antineoplastic drug (carboplatin) increased pCR rates in the triple-negative subgroup to 51%, versus 26% in the control group (Rugo et al., 2016. New Engl J Med 375: 23-34). Additional data showed that a 7-gene DNA repair deficiency expression signature (PARPi-7; Daemen et al., 2012. Breast Cancer Res Treat 135: 505-517), BRCAlness and MammaPrint® signatures may help refine predictions of VC response, thereby improving patient care (Van't Veer et al., 2015. J Clin Oncol 33: 521-521; Wolf et al., 2017. NPJ Breast Cancer 3: 31). In addition, the immune check point inhibitor pembrolizumab, when added to standard neoadjuvant chemotherapy, more than doubled the estimated pCR rates for both ERBB2-negative (HER2-negative) breast cancer (Nanda et al., 2020. JAMA Oncology: doi: 10.1001/jamaonco1.2019.6650).

However, there is a need for new treatment options, including new combinations of agents, that could provide therapeutic benefit for specific cancer patients, especially Her2 negative breast cancer patients. Additionally, there is a need to identify cancer patients that might benefit from new treatment options, including new combinations of agents.

### 2 BRIEF DESCRIPTION OF THE INVENTION

The invention provides a poly [ADP-ribose] polymerase (PARP) inhibitor, for use in a method of treating a human epidermal growth factor receptor 2 (HER2) negative, MammaPrint high risk 2 (MP2) breast cancer. Said PARP inhibitor for use preferably is selected from olaparib, rucaparib, pamiparib, niraparib and talazoparib.

The invention further provides an immune check point inhibitor, for use in a method of treating a human epidermal growth factor receptor 2 (HER2) negative, MammaPrint high risk 2 (MP2) breast cancer. Said immune check point inhibitor for use preferably is selected from tremelimumab, pembrolizumab, nivolumab, pidilizumab, cemiplimab, atezolizumab, avelumab and durvalumab.

The invention further provides combination of a poly [ADP-ribose] polymerase (PARP) inhibitor and an immune check point inhibitor for use in a method of treating a human epidermal growth factor receptor 2 (HER2) negative, MammaPrint high2 breast cancer. Said PARP inhibitor is selected from olaparib, rucaparib, pamiparib, niraparib and talazoparib. Said immune check point inhibitor preferably is selected from tremelimumab, pembrolizumab, nivolumab, pidilizumab, cemiplimab, atezolizumab, avelumab and durvalumab. Said PARP inhibitor is administrated simultaneously with, separately from, or sequentially to the immune check point inhibitor.

A method of treating according to the invention preferably further comprises administration of a taxane, preferably selected from paclitaxel, docetaxel, and cabazitaxel.

Said HER2 negative breast cancer preferably is Estrogen Receptor (ER) positive, preferably Hormone Receptor (HR) positive.

A HER2 status is preferably determined by TargetPrint or by BluePrint.

The invention further provides a method of treating a HER2 negative, MammaPrint high risk 2 (MP2) breast cancer in a subject, comprising the simultaneous, separate or sequential administering to the subject of a PARP inhibitor and an immune check point inhibitor. Said method preferably further comprises administering a taxane, preferably selected from paclitaxel, docetaxel, and cabazitaxel.

The invention further provides a pharmaceutical composition comprising a PARP inhibitor and an immune check point inhibitor, for use in a method of treating a human epidermal growth factor receptor 2 (HER2) negative, MammaPrint high risk 2 (MP2) breast cancer. Said method of treating preferably further comprises administering a taxane, preferably selected from paclitaxel, docetaxel, and cabazitaxel.

### 3 BRIEF DESCRIPTION OF THE FIGURES

FIG. 1. pCR probability by signature. Combined Duravalumab+Olaparib graduated in all 3 eligible biomarker signatures HR+/HER2− (A), HR−/HER2− (B), and combined HER2− (C) by demonstrating increased pCR.

FIG. 2. Combined Durvalumab/Olaparib treatment in addition to standard chemotherapy shifted residual cancer burden (RCB) to lower values across all RCB categories in all MammaPrint high risk samples in HER2 negative subtypes.

FIG. 3. MammaPrint high risk group 2 (MP2) drives the benefit in the ER+/HER2− subtype (HR+HER2−). MP2 is defined as >median MammaPrint score (Wolf et al., 2017. NPJ Breast Cancer 3: 1-9). HR+/HER2−/MP1: n=132 (D:24; Ctr: 108). HR+/HER2−/MP2: n=77 (D:28; Ctr: 49).

### 4 DETAILED DESCRIPTION OF THE INVENTION

### 4.1 Definitions

As is used herein, the term “Poly [ADP-ribose] polymerase (PARP) inhibitor”, an refers to an inhibitor of a poly [ADP-ribose] polymerase. PARP is a key factor in the initiation of a repair response to single-strand DNA breaks (SSB). A preferred PARP inhibitor is selective for PARP1 and/or PARP2, when compared to other polymerases, meaning that the inhibitor is at least two times more potent, preferably at least five times more potent, in inhibiting PARP, when compared to other polymerases.

As is used herein, the term “immune checkpoint inhibitor”, refers to an inhibitor of an immune checkpoint molecule, a regulator of the immune system. Immune checkpoint molecules include CTLA4, PD-1 and PD-L1, A2AR, CD276, B7-H4, CD272 and Herpesvirus Entry Mediator (HVEM), LAG3, NOX2, TIM-3, V-domain Ig suppressor of T cell activation (VISTA), and CD328. A preferred immune checkpoint inhibitor is selective for at least one of CTLA4, PD-1 and PD-L1, A2AR, CD276, B7-H4, CD272 and Herpesvirus Entry Mediator (HVEM), LAG3, NOX2, TIM-3, V-domain Ig suppressor of T cell activation (VISTA), and CD328, when compared to other surface molecules, meaning that the inhibitor is at least two times more potent, preferably at least five times more potent, in inhibiting at least one of CTLA4, PD-1 and PD-L1, A2AR, CD276, B7-H4, CD272 and Herpesvirus Entry Mediator (HVEM), LAG3, NOX2, TIM-3, V-domain Ig suppressor of T cell activation (VISTA), and CD328, when compared to other molecules.

As is used herein, the term “combination” refers to the administration of effective amounts of a PARP inhibitor and an immune checkpoint inhibitor to a patient in need thereof. Said PARP inhibitor and immune checkpoint inhibitor may be provided in one pharmaceutical preparation, or as two distinct pharmaceutical preparations.

As is used herein, the term “taxane”, also termed taxoid, refers to a dipertene that disrupts microtubule function by stabilizing GDP-bound tubulin in a microtubule. This inhibits depolymerization of the microtubule, which is required during cell division. A preferred taxane is selected from paclitaxel, docetaxel, and cabazitaxel.

As is used herein, the term “carcinoma” refers to a cancer that has an epithelial origin,

As is used herein, the term “HER2-negative breast cancer, or HER2−” refers to a breast cancer that does not detectably express human epidermal growth factor receptor 2 (HER2). Similarly, the term “HER2-positive breast cancer or HER2+” refers to a breast cancer that does detectably express HER2. HER2 is also termed v-erb-b2 avian erythroblastic leukemia viral oncogene homolog 2 (ERBB2) or NEU.

As is used herein, the term “ER-negative breast cancer or ER-” refers to a breast cancer that does not detectably express estrogen receptor (ER). Similarly, the term “ER-positive breast cancer or ER+” refers to a breast cancer that does detectably express estrogen receptor (ER).

As is used herein, the term “PR-negative breast cancer or PR−” refers to a breast cancer that does not detectably express progesterone receptor (PR). Similarly, the term “PR-positive breast cancer or PR+” refers to a breast cancer that does detectably express PR.

As is used herein, the term “HR-negative breast cancer or HR−” refers to a breast cancer that does not detectably express estrogen receptor (ER) and progesterone receptor (PR). Similarly, the term “HR-positive breast cancer or HR+” refers to a breast cancer that does detectably express ER and PR.

As is used herein, the term “TargetPrint®” refers to a quantitative mRNA expression level testing assay for ER, PR, and HER2 status of breast cancer (Wesseling et al., 2016. Virchows Archiv 469: 297-304).

As is used herein, the term “BluePrint®” (U.S. Pat. Nos. 9,175,351; 10,072,301; Krijgsman et al., 2012. Br Can Res Treatm 133: 37-47) refers to a molecular subtyping test, analyzing the activity of 80 genes to enable stratification of a breast cancer into one of the three following subtypes: Luminal-type, HER2− type and Basal-type.

The term “RNA expression product”, as used herein, refers to an expression product of a gene and includes gene expression products such as RNA, including mRNA. Also included in this term are complementary nucleic acids derived from a RNA gene expression product, such as cDNA and cRNA. Preferably, the gene expression products in a sample from a cancer patient are RNA expression products, including mRNA, cDNA and cRNA.

### 4.2 Methods of Diagnosis

A gene signature test (MammaPrint®, MP), also termed “Amsterdam gene signature test” determines the expression of gene expression products of signature genes and stratifies early-stage breast cancer patients in Low- and High risk for developing distant metastases within 5 years after diagnosis. Extensive validation studies (Drukker et al., 2013. Int J Cancer 133: 929-936; Bueno-de-Mesquita et al., 2007. Lancet Oncol 8: 1079-1087; van de Vijver et al., 2002. New Engl J Med 34: 1999-2009) and the recent MINDACT clinical trial (Cardoso et al., 2016. N Engl J Med 375: 717-729) have demonstrated the clinical utility of MammaPrint (level 1A clinical evidence), making it a unique example of a clinical diagnostic test that helps guide physicians in adjuvant treatment decisions for breast cancer patients.

A sample from a cancer patient comprising gene expression products from a cancer cell of said patient can be obtained in numerous ways, as is known to a person skilled in the art. In a method of the invention, a sample can be obtained directly from the individual, for example by taking a biopsy from the cancer.

Said sample may also be obtained from a breast cancer after removal of the cancer, or at least part of the cancer, from a patient. Said sample is preferably obtained from a cancer within two hours after removal, more preferably within 1 hour after removal of the cancer or part of the cancer.

Before a sample comprising gene expression products is obtained from a cancer, said cancer may be cooled and stored at about 0-8° C. The sample can be freshly prepared from cells or a tissue sample at the moment of harvesting, or they can be prepared from samples that are stored at −70° C. until processed for sample preparation.

Alternatively, tissues or biopsies can be stored under conditions that preserve the quality of protein or RNA. Examples of these preservative conditions are fixation using e.g. formaline and paraffin embedding, RNase inhibitors such as RNAsin (Pharmingen) or RNasecure (Ambion), aquous solutions such as RNAlater (Assuragen; U.S. Ser. No. 06/204,375), Hepes-Glutamic acid buffer mediated Organic solvent Protection Effect (HOPE; DE10021390), and RCL2 (Alphelys; WO04083369), and non-aquous solutions such as Universal Molecular Fixative (Sakura Finetek USA Inc.; U.S. Pat. No. 7,138,226). Alternatively, a sample from a cancer patient may be fixated in formalin, for example as formalin-fixed paraffin-embedded (FFPE) tissue. Preferably, the sample is an FFPE sample.

Methods to determine gene expression levels of genes are known to a skilled person and include, but are not limited to, Northern blotting, quantitative PCR, microarray analysis and RNA sequencing.

It is preferred that said gene expression levels are determined simultaneously. Simultaneous analyses can be performed, for example, by multiplex qPCR, RNA sequencing procedures, and microarray analysis. Microarray analysis allow the simultaneous determination of gene expression levels of expression of a large number of genes, such as more than 50 genes, more than 100 genes, more than 1000 genes, more than 10.000 genes, or even whole-genome based, allowing the use of a large set of gene expression data for normalization of the determined gene expression levels in a method of the invention.

Microarray-based analysis involves the use of selected biomolecules that are immobilized on a solid surface, an array. A microarray usually comprises nucleic acid molecules, termed probes, which are able to hybridize to gene expression products. The probes are exposed to labeled sample nucleic acid, hybridized, and the abundance of gene expression products in the sample that are complementary to a probe is determined. The probes on a microarray may comprise DNA sequences, RNA sequences, or copolymer sequences of DNA and RNA. The probes may also comprise DNA and/or RNA analogues such as, for example, nucleotide analogues or peptide nucleic acid molecules (PNA), or combinations thereof. The sequences of the probes may be full or partial fragments of genomic DNA. The sequences may also be in vitro synthesized nucleotide sequences, such as synthetic oligonucleotide sequences.

In the context of the invention, a probe is to be specific for a gene expression product of a gene as listed in Table 1. A probe is specific when it comprises a continuous stretch of nucleotides that are completely complementary to a nucleotide sequence of a gene expression product, or a cDNA product thereof. A probe can also be specific when it comprises a continuous stretch of nucleotides that are partially complementary to a nucleotide sequence of a gene expression product of said gene, or a cDNA product thereof. Partially means that a maximum of 5% from the nucleotides in a continuous stretch of at least 20 nucleotides differs from the corresponding nucleotide sequence of a gene expression product of said gene. The term complementary is known in the art and refers to a sequence that is related by base-pairing rules to the sequence that is to be detected. It is preferred that the sequence of the probe is carefully designed to minimize nonspecific hybridization to said probe.

It is preferred that the probe is, or mimics, a single stranded nucleic acid molecule. The length of said complementary continuous stretch of nucleotides can vary between 15 bases and several kilo bases, and is preferably between 20 bases and 1 kilobase, more preferred between 40 and 100 bases, and most preferred about 60 nucleotides. A most preferred probe comprises about 60 nucleotides that are identical to a nucleotide sequence of a gene expression product of a gene, or a cDNA product thereof. In a method of the invention, probes comprising probe sequences as indicated in Tables 1-2 can be employed.

To determine the RNA expression level by micro-arraying, gene expression products in the sample are preferably labeled, either directly or indirectly, and contacted with probes on the array under conditions that favor duplex formation between a probe and a complementary molecule in the labeled gene expression product sample. The amount of label that remains associated with a probe after washing of the microarray can be determined and is used as a measure for the gene expression level of a nucleic acid molecule that is complementary to said probe.

The determined RNA expression level can be normalized for differences in the total amounts of nucleic acid expression products between two separate samples by comparing the level of expression of one or more genes that are known not to differ in expression level between samples. If samples for use in a method of the invention are FFPE samples, it is possible to use an FFPE normalization template.

A preferred method for determining RNA expression is by microarray analysis.

Another preferred method for determining RNA expression levels is by sequencing, preferably next-generation sequencing (NGS), of RNA samples, with or without prior amplification of the RNA expression products.

High throughput sequencing techniques for sequencing RNA have been developed. NGS platforms, including Illumina® sequencing; Roche 454 Pyrosequencing®, ion torrent and ion proton sequencing, and ABI SOLID® sequencing, allow sequencing of fragments of DNA in parallel. Bioinformatics analyses are used to piece together these fragments by mapping the individual reads. Each base is sequenced multiple times, providing high depth to deliver accurate data and an insight into unexpected DNA variation. NGS can be used to sequence a complete exome including all or small numbers of individual genes.

Pyrosequencing detects the release of inorganic pyrophosphate (PPi) as particular nucleotides are incorporated into the nascent strand (Ronaghi et al., 1996. Analytical Biochemistry 242: 84-9; Ronaghi, 2001. Genome Res 11: 3-11; Ronaghi et al., 1998. Science 281: 363; U.S. Pat. Nos. 6,210,891; 6,258,568; and 6,274,320, which are all incorporated herein by reference. In pyrosequencing, released PPi can be detected by being immediately conversion to adenosine triphosphate (ATP) by ATP sulfurylase, and the level of ATP generated is detected via luciferase-produced photons.

NGS also includes so called third generation sequencing platforms, for example nanopore sequencing on an Oxford Nanopore Technologies platform, and single-molecule real-time sequencing (SMRT sequencing) on a PacBio platform, with or without prior amplification of the RNA expression products.

Further high throughput sequencing techniques include, for example, sequencing-by-synthesis. Sequencing-by-synthesis or cycle sequencing can be accomplished by stepwise addition of nucleotides containing, for example, a cleavable or photobleachable dye label as described, for example, in U.S. Pat. Nos. 7,427,673; 7,414,116; WO 04/018497; WO 91/06678; WO 07/123744; and U.S. Pat. No. 7,057,026, all of which are incorporated herein by reference.

Sequencing techniques also include sequencing by ligation techniques. Such techniques use DNA ligase to incorporate oligonucleotides and identify the incorporation of such oligonucleotides and are inter alia described in U.S. Pat. Nos. 6,969,488; 6,172,218; and 6,306,597. Other sequencing techniques include, for example, fluorescent in situ sequencing (FISSEQ), and Massively Parallel Signature Sequencing (MPSS).

Sequencing techniques can be performed by directly sequencing RNA, or by sequencing a RNA-to-cDNA converted nucleic acid library. Most protocols for sequencing RNA samples employ a sample preparation method that converts the RNA in the sample into a double-stranded cDNA format prior to sequencing. Conversion of RNA into cDNA and/or cRNA using a reverse-transcriptase enzyme such as M-MLV reverse-transcriptase from Moloney murine leukemia virus, or AMV reverse-transcriptase from avian myeloblastosis virus, is known to a person skilled in the art.

In the methods of diagnosing, the reference sample preferably is a sample, such as an RNA sample, isolated from a tissue of a healthy individual, or isolated from a cancerous growth of a cancer patient, preferably a breast cancer patient. The reference sample may comprise an RNA sample from a relevant cell line or mixture of cell lines. The RNA from a cell line or cell line mixture can be produced in-house or obtained from a commercial source such as, for example, Stratagene Human Reference RNA. Another preferred reference sample comprises RNA isolated and pooled from normal adjacent tissue from cancer patients.

Even more preferably, said reference sample is a pooled RNA sample that is isolated from tissue comprising cancer cells from multiple individuals suffering from cancer, preferably breast cancer, more preferably stage 2 and/or 3 breast cancer, and which cancer cells either have an activated or not activated PD1. It is preferred that said sample is pooled from more than 10 individuals, more preferred more than 20 individuals, more preferred more than 30 individuals, more preferred more than 40 individuals, most preferred more than 50 individuals.

Typing of a sample can be performed in various ways. In one method, a coefficient is determined that is a measure of a similarity or dissimilarity of a sample with a previously established reference RNA expression level of the target genes that is specific to a certain cell type, tissue, disease state or any other interesting biological or clinical interesting samples group. Such a reference RNA expression level can be referred to as a profile template. Typing of a sample can be based on its (dis)similarity to a single profile template or based on multiple profile templates. In the invention, the profile templates are representative of samples that have low risk outcome, high risk outcome, or both low risk and high risk outcome. Examples of suitable profile templates are RNA expression level templates of a single breast cancer sample from a patient with low or high risk outcome, preferably a group of at least 10, 30, 40, 50, 100, 200 or 300 breast cancer patients with low and/or high risk outcome.

A number of different coefficients can be used for determining a correlation between the gene expression level in a sample from a cancer patient and a profile template. Preferred methods are parametric methods which assume a normal distribution of the data. One of these methods is the Pearson product-moment correlation coefficient, which is obtained by dividing the covariance of the two variables by the product of their standard deviations. Preferred methods comprise cosine-angle, un-centered correlation and, more preferred, cosine correlation (Fan et al., Conf Proc IEEE Eng Med Biol Soc. 5:4810-3 (2005)).

Said correlation with a profile template is used to produce an overall similarity score for the set of genes that are used. A similarity score is a measure of the average correlation of gene expression levels of a set of genes in a sample from a cancer patient and a profile template. Said similarity score can, but does not need to be, a numerical value between +1, indicative of a high correlation between the gene expression level of the set of genes in a sample of said cancer patient and said profile template, and −1, which is indicative of an inverse correlation. A threshold can be used to differentiate between samples having low risk outcome, and samples having high risk outcome. Said threshold is an arbitrary value that allows for discrimination between samples from patients with low risk outcome, and samples of patients with high risk outcome. If a similarity threshold value is employed, it is preferably set at a value at which an acceptable number of patients with high risk outcome would score as false negatives, and an acceptable number of patients with low risk outcome would score as false positives. A similarity score is preferably displayed or outputted to a user interface device, a computer readable storage medium, or a local or remote computer system.

A method of typing of the invention may further comprise determining a stage of the cancer. The staging of a cancer is generally based on the size of the cancer and on whether the cancer has spread to lymph nodes or other areas of the body.

A preferred staging system is the TNM (for tumors/nodes/metastases) system, from the American Joint Committee on Cancer (AJCC). The TNM system assigns a number based on three categories. “T” denotes the size of the tumor, “N” the degree of lymphatic node involvement, and “M” the degree of metastasis.

The methods of assigning a PARP inhibitor, an immune checkpoint inhibitor, and a combination of a PARP inhibitor and an immune checkpoint inhibitor to a HER2 negative breast cancer patient further comprise determining a level of RNA expression for a plurality of genes consisting of at least 5 of the genes for which markers are listed in Table 1 in a sample comprising RNA expression products from a cancer cell of the patient.

MammaPrint® (MP) is a 70 genes-based assay, as described in WO2002103320, which is hereby incorporated by reference. MP reports a risk of cancer recurrence within a period of 5 years without adjuvant chemotherapy. MP is intended to classify an individual suffering from breast cancer as having a good prognosis having no distant metastases within five years of initial diagnosis (low risk outcome), or as having a poor prognosis having distant metastases within five years of initial diagnosis (high risk outcome).

MP was shown to successfully predict metastasis free survival and overall survival in retrospective and prospective studies (van de Vijver et al., 2002. N Engl J Med 347: 1999-2009; van't Veer et al., 2002. Nature 415: 530-536; Drukker et al., 2013. Int J Cancer 133: 929-936; Cardoso et al., 2016. N Engl. J Med 375: 717-729).

The MammaPrint high risk group can be further divided into 2 groups by taking the median MammaPrint index of the high risk samples. MammaPrint high risk group 2 (MP2) is defined as having a MammaPrint index higher than the median value, while MP1 is defined as having a MammaPrint index lower than the median value

It was now surprisingly found that MP may also be used to predict a response to a PARP inhibitor, an immune checkpoint inhibitor, and to a combination of a PARP inhibitor and an immune checkpoint inhibitor of a HER2 negative breast cancer patient. More specifically, an individual suffering from a HER2 negative breast cancer and typed as having MammaPrint high risk group 2 (MP2), thus with a poor prognosis, is likely to provide a pathological complete response (pCR) upon treatment with mediated therapy.

A method of the invention for predicting a response to a PARP inhibitor, an immune checkpoint inhibitor, or to the combination thereof, of HER2-negative breast cancer involves the use of at least 5 genes indicated in Table 1, more preferred at least 6 genes, more preferred at least 7 genes, more preferred at least 8 genes, more preferred at least 9 genes, more preferred at least 10 genes, more preferred at least 20 genes, more preferred at least 30 genes, more preferred at least 40 genes, more preferred at least 50 genes, more preferred at least 60 genes, more preferred at least 70 genes indicated in Table 1, such as all 231 genes listed in Table 1.

The set of genes selected from the genes listed in Table 1 preferably contains at least the first 5, 10, 15, 20, 25, 30, 35, 40, 45, 50, 55, 60, 65, 70, 75, 80, 85, 90, 95 or 100 rank-ordered genes of Table 1. The genes in Table 1 were rank-ordered according to the agreement of the outcome of typing of a sample with the individual genes to the outcome of the typing of a sample with the set of genes listed in Table 2. A preferred set of genes comprises both positively correlated genes as well as negatively correlated genes, as indicated in Table 1, whereby said correlation is to a good prognosis signature.

A further preferred set of genes according to the invention comprises at least five genes of Table 1 that are rank-ordered 1-5 and/or 227-231. A further preferred set of genes according to the invention comprises at least ten genes of Table 1 that are rank-ordered 1-10 and/or 222-231, more preferred at least twenty genes listed in Table 1 that are rank-ordered 1-20 and/or 212-231; more preferred at least fifty genes listed in Table 1 that are rank-ordered 1-50 and/or 182-231; more preferred at least hundred genes listed in Table 1 that are rank-ordered 1-100 and/or 132-231; more preferred all 231 genes listed in Table 1.

A preferred set of genes for predicting a response to a PARP inhibitor, an immune checkpoint inhibitor, or to the combination thereof, of HER2-negative breast cancer involves the use of a subset of 70 genes, which are indicated in Table 2 and for which preferred probes are provided in Table 2.

A further preferred method of the invention for predicting a response to a PARP inhibitor, an immune checkpoint inhibitor, or to the combination thereof, of HER2-negative breast cancer involves the use of at least 5 genes indicated in Table 2, more preferred at least 6 genes, more preferred at least 7 genes, more preferred at least 8 genes, more preferred at least 9 genes, more preferred at least 10 genes, more preferred at least 20 genes, more preferred at least 30 genes, more preferred at least 40 genes, more preferred at least 50 genes, more preferred at least 60 genes, more preferred all 70 genes indicated in Table 2.

A further preferred set of genes according to the invention comprises at least five genes of Table 2 that are rank-ordered 1-5 and/or 66-70. A further preferred set of genes according to the invention comprises at least ten genes of Table 2 that are rank-ordered 1-10 and/or 61-70, more preferred at least twenty genes listed in Table 2 that are rank-ordered 1-20 and/or 51-70; more preferred at least fifty genes listed in Table 2 that are rank-ordered 1-50 and/or 21-70; more preferred all 70 genes listed in Table 2.

It is noted that some probes hybridize to the same genes indicated in Table 2, such as three probes which are now known to hybridize to expression products of the Diaphanous Related Formin 3 (DIAPH3; ENSG00000139734) gene. A reference to different genes listed in Table 2 includes reference to different probes hybridizing to the same gene listed in Table 2. Hence, the term “at least five genes of Table 2” provides reference to both 5 different genes listed in Table 2 as well as 5 different probes listed in Table 2.

A method of the invention for predicting a response to a PARP inhibitor, an immune checkpoint inhibitor, or to the combination thereof, of HER2-negative breast cancer is especially suited if the breast cancer is positive for Estrogen Receptor (ER), and/or Progesteron Receptor (PR), collectively termed Hormone Receptors (HR).

ER, PR and HER/HER2 status may be determined, for example, by ImmunoHistoChemistry (IHC), by TargetPrint (McShane et al., 2005. J Clin Oncol 23: 9067-72; Roepman et al., 2009. Clin Cancer Res 15: 7004-70115), and/or by BluePrint (U.S. Pat. Nos. 9,175,351; 10,072,301; Krijgsman et al., 2012. Br Can Res Treatm 133: 37-47), as is known to a person skilled in the art.

### 4.3 Methods of Treatment

The invention provides a PARP inhibitor for use as a medicament for the treatment of a HER2 negative, MammaPrint high risk 2, breast cancer patient.

Further provided is an immune checkpoint inhibitor for use as a medicament for the treatment of a HER2 negative, MammaPrint high risk 2, breast cancer patient.

Further provided is a PARP inhibitor for use as a medicament for the treatment of a HER2 negative, MammaPrint high risk 2, breast cancer patient, wherein said medicament further comprises an immune checkpoint inhibitor.

Further provided is an immune checkpoint inhibitor for use as a medicament for the treatment of a HER2 negative, MammaPrint high risk 2, breast cancer patient, wherein said medicament further comprises a PARP inhibitor.

Further provided is a combination of a PARP inhibitor and an immune checkpoint inhibitor, for use as a medicament for the treatment of a HER2 negative, MammaPrint high risk 2, breast cancer patient.

Further provided is a PARP inhibitor for use as a medicament for the treatment of a HER2 negative, MammaPrint high risk 2, breast cancer patient, wherein said medicament further comprises an immune checkpoint inhibitor.

Further provided is a combination of a PARP inhibitor and an immune checkpoint inhibitor, for use as a medicament for the treatment of a HER2 negative, MammaPrint high risk 2, breast cancer patient.

Further provided is a method of treating an individual suffering from a HER2 negative, MammaPrint high risk 2, breast cancer, comprising providing said individual with a PARP inhibitor and an immune checkpoint inhibitor.

Further provided is an use of a PARP inhibitor in the preparation of a medicament for treatment of a HER2 negative, MammaPrint high risk 2, breast cancer patient, wherein said treatment further comprises an immune checkpoint inhibitor.

Further provided is an use of an immune checkpoint inhibitor in the preparation of a medicament for treatment of a HER2 negative, MammaPrint high risk 2, breast cancer patient, wherein said treatment further comprises a PARP inhibitor.

Further provided is a combination of a PARP inhibitor and an immune checkpoint inhibitor, for use in the treatment of a HER2 negative, MammaPrint high risk 2, breast cancer patient.

The invention further provides the use of a PARP inhibitor in the preparation of a medicament for the treatment of a HER2 negative, MammaPrint high risk 2, breast cancer patient.

Further provided is the use of an immune checkpoint inhibitor in the preparation of a medicament for the treatment of a HER2 negative, MammaPrint high risk 2, breast cancer patient.

Further provided is the use of a PARP inhibitor in the preparation of a medicament for the treatment of a HER2 negative, MammaPrint high risk 2, breast cancer patient, wherein said medicament further comprises an immune checkpoint inhibitor.

Further provided is the use of an immune checkpoint inhibitor in the preparation of a medicament for the treatment of a HER2 negative, MammaPrint high risk 2, breast cancer patient, wherein said medicament further comprises a PARP inhibitor.

Further provided is the use of a combination of a PARP inhibitor and an immune checkpoint inhibitor, in the preparation of a medicament for the treatment of a HER2 negative, MammaPrint high risk 2, breast cancer patient.

Further provided is a method of treating an individual suffering from a HER2 negative, MammaPrint high risk 2, breast cancer, comprising providing said individual with a PARP inhibitor.

Further provided is a method of treating an individual suffering from a HER2 negative, MammaPrint high risk 2, breast cancer, comprising providing said individual with an immune checkpoint inhibitor.

Further provided is a method of treating an individual suffering from a HER2 negative breast cancer that has a high risk for recurrence, comprising providing said individual with a PARP inhibitor, an immune checkpoint inhibitor, or a combination of a PARP inhibitor and an immune checkpoint inhibitor, optionally in combination with a taxane. A person skilled in the art is able to determine a high risk for recurrence, for example by employing Oncotype DX (Genomic Health), EndoPredict (Myriad Genetics, Inc.), Breast Cancer Index (bioTheranostics) or Prosigna Breast Cancer Prognostic Gene Signature Assay (NanoString Technologies).

A PARP inhibitor preferably is selected from olaparib (3-aminobenzamide, 4-(3-(1-(cyclopropanecarbonyl)piperazine-4-carbonyl)-4-fluorobenzyl)phthalazin-1(2H)-one; AZD-2281; AstraZeneca), rucaparib (6-fluoro-2-[4-(methylaminomethyl)phenyl]-3,10-diazatricyclo[6.4.1.04,13]trideca-1,4,6,8(13)-tetraen-9-one; Clovis Oncology, Inc.); niraparib tosylate ((S)-2-(4-(piperidin-3-yl)phenyl)-2H-indazole-7-carboxamide hydrochloride; MK-4827; GSK); talazoparib (11S,12R)-7-fluoro-11-(4-fluorophenyl)-12-(2-methyl-1,2,4-triazol-3-yl)-2,3,10-triazatricyclo[7.3.1.05,13]trideca-1,5(13),6,8-tetraen-4-one; BMN-673; Pfizer); veliparib (2-[(2R)-2-methylpyrrolidin-2-yl]-1H-benzimidazole-4-carboxamide dihydrochloride benzimidazole carboxamide; ABT-888; Abbvie); pamiparib (2R)-14-fluoro-2-methyl-6,9,10,19-tetrazapentacyclo[14.2.1.02,6.08,18.012,17]nonadeca-1(18),8,12(17),13,15-pentaen-11-one; BGB-290; BeiGene); CEP-8983, and CEP 9722, a small-molecule prodrug of CEP-8983, a 4-methoxy-carbazole inhibitor (CheckPoint Therapeutics); E7016 (Eisai), PJ34 (2-(dimethylamino)-N-(6-oxo-5H-phenanthridin-2-yl)acetamide;hydrochloride) and 3-aminobenzamide.

A preferred PARP inhibitor is selected from the group consisting of olaparib, rucaparib, niraparib tosylate, talazoparib, and pamiparib.

Said PARP inhibitor preferably is administered orally, as a tablet or as a capsule. Said PARP inhibitor preferably is administered once or twice per day for a period of 1-24 weeks, for example once or twice daily for a 12 weeks period. The preferred dosage of selected PARP inhibitors is 100-500 mg twice daily, preferably 300-400 mg twice daily for olaparib; 200-1000 mg twice daily, preferably 400-600 mg twice daily for rucaparib; 50-500 mg twice daily, preferably 100-300 mg twice daily for niraparib tosylate; 0.2-2 mg twice daily, preferably 0.5-1 mg twice daily for talazoparib; 100-600 mg twice daily, preferably 200-400 mg twice daily for veliparib; and 300-100 mg twice daily, preferably 40-60 mg twice daily for pamiparib. A person skilled in the art will understand that the dosage in a combination according to the invention, may be at the low range of the indicated dosages, or even below the indicated dosages.

An immune checkpoint inhibitor is an inhibitor of CTLA4, PD-1 and PD-L1, A2AR, CD276, B7-H4, CD272 and Herpesvirus Entry Mediator (HVEM), LAG3, NOX2, TIM-3, V-domain Ig suppressor of T cell activation (VISTA), and CD328. Said inhibitor preferably is a PD1/PDL1 inhibitor and/or an inhibitor of CTLA-4. Suitable immune checkpoint inhibitors are CTLA-4 inhibitors such as antibodies, including ipilimumab (Bristol-Myers Squibb) and tremelimumab (MedImmune); PD1/PDL1 inhibitors such as antibodies, including pembrolizumab (Merck), nivolumab and MDX-1105 (Bristol-Myers Squibb), pidilizumab (Medivation/Pfizer), MEDI0680 (AMP-514; AstraZeneca), cemiplimab (Regeneron) and PDR001 (Novartis); fusion proteins such as a PD-L2 Fc fusion protein (AMP-224; GlaxoSmithKline); atezolizumab (Roche/Genentech), avelumab (Merck/Serono and Pfizer), durvalumab (AstraZeneca), BMS-936559 (Bristol-Myers Squibb); and small molecule inhibitors such as PD-1/PD-L1 Inhibitor 1 (WO2015034820; (2S)-1-[[2,6-dimethoxy-4-[(2-methyl-3-phenylphenyl)methoxy]phenyl]methyl]piperidine-2-carboxylic acid), BMS202 (PD-1/PD-L1 Inhibitor 2; WO2015034820; N-[2-[[[2-methoxy-6-[(2-methyl[1,1′-biphenyl]-3-yl)methoxy]-3-pyridinyl]methyl]amino]ethyl]-acetamide), PD-1/PD-L1 Inhibitor 3 (WO/2014/151634; (3S,6S,12S,15S,18S,21S,24S,27S,30R,39S,42S,47aS)-3-((1H-imidazol-5-yl)methyl)-12,18-bis((1H-indol-3-yl)methyl)-N,42-bis(2-amino-2-oxoethyl)-36-benzyl-21,24-dibutyl-27-(3-guanidinopropyl)-15-(hydroxymethyl)-6-isobutyl-8,20,23,38,39-pentamethyl-1,4,7,10,13,), and ladiratuzumab vedotin (Seattle Genetics).

Said immune checkpoint inhibitor preferably is administered intravenously, preferably by infusion. Said immune checkpoint inhibitor preferably is administered once every 2-4 weeks for a period of 1-24 weeks. The preferred dosage of selected immune checkpoint inhibitors is 2-4 mg/kg. preferably about 3 mg/kg every 2-4 weeks, or 240-480 mg every 2-4 weeks for ipilimumab; 100-400 mg, preferably about 200 mg every 2-4 weeks, preferably every 3 weeks for pembrolizumab; 100-500 mg, preferably 240-480 mg every 2-4 weeks, preferably every 2 weeks for nivolumab; 2-12 mg/kg. preferably 4-8 mg/kg every 2-4 weeks, preferably every 4 weeks for pidilizumab; 100-500 mg, preferably about 350 mg every 2-4 weeks, preferably every 3 weeks for cemiplimab; 600-1800 mg, preferably about 1200 mg every 2-4 weeks, preferably every 3 weeks for atezolizumab; 400-1200 mg, preferably about 800 mg, every 2-4 weeks, preferably every 2 weeks for avelumab; and 5-15 mg/kg, preferably about 10 mg/kg, or 1000-2000 mg, preferably about 1500 mg, every 2-4 weeks, preferably every 2 weeks for durvalumab. A person skilled in the art will understand that the dosage in a combination according to the invention, may be at the low range of the indicated dosages, or even below the indicated dosages.

In a combination according to the invention, a PARP inhibitor is administrated simultaneously with, separately from, or sequentially to the immune checkpoint inhibitor. When administered as two distinct pharmaceutical preparations, they may be administered on the same day or on different days to a patient in need thereof, and using a similar or dissimilar administration protocol, e.g. daily, twice daily, biweekly, orally and/or by infusion. For example, said PARP inhibitor may be administrated at a daily basis, while the immune checkpoint inhibitor may be administered at a weekly basis, for example every 2 weeks or every 4 weeks.

Said combination is preferably administered repeatedly according to a protocol that depends on the patient to be treated (age, weight, treatment history, etc.), which can be determined by a skilled physician. Said protocol may include daily or weekly administration for 1-30 days, such as 2 days, 10 days, 21 days, or 28 days, followed by period of 0-14 days, such as 7 days in which no inhibitor is administered. As an alternative, said daily or weekly administration may be followed by another therapy, for example a combination of doxorubicin (60 mg/m²) and cyclophosphamide (600 mg/m²) for 4 cycles every 3 weeks.

A combination comprising a PARP inhibitor and an immune checkpoint inhibitor according to the invention may further be combined with a taxane. Said taxane preferably is administered in the same time frame as the PARP inhibitor and the immune checkpoint inhibitor are administered, for example in a period of 1-30 days, such as 2 days, 10 days, 21 days, or 28 days.

Said taxane preferably is administered intravenously, preferably by infusion. Said taxane preferably is repeatedly administered, for example once every week, once every two weeks, or once every three weeks. For example, paclitaxel may be administered at a dosage of 75-200 mg/m², such as about 80 mg/m², every 1-4 weeks; docetaxel may be administered at a dosage of 40-100 mg/m², such as about 60 mg/m², every 1-4 weeks; and cabazitaxel may be administered at a dosage of 5-75 mg/m², such as about 20 mg/m², every 1-4 weeks.

### 4.4 Compositions

A combination of a PARP inhibitor and an immune checkpoint inhibitor according to the invention may be provided in one pharmaceutical preparation or, preferably, as two or more distinct pharmaceutical preparations. Said distinct pharmaceutical preparations may further comprise pharmaceutically acceptable excipients, as is known to a person skilled in the art.

Pharmaceutically acceptable excipients include diluents, binders or granulating ingredients, a carbohydrate such as starch, a starch derivative such as starch acetate and/or maltodextrin, a polyol such as xylitol, sorbitol and/or mannitol, lactose such as α-lactose monohydrate, anhydrous α-lactose, anhydrous β-lactose, spray-dried lactose, and/or agglomerated lactose, a sugar such as dextrose, maltose, dextrate and/or inulin, or combinations thereof, glidants (flow aids) and lubricants to ensure efficient tableting, and sweeteners or flavors to enhance taste.

The invention therefore provides a pharmaceutical composition, comprising a PARP inhibitor and an immune checkpoint inhibitor for use in a method of treating a patient suffering from a HER2 negative, MammaPrint high risk 2 (MP2) breast carcinoma. Said HER2 negative, MammaPrint high risk 2 (MP2) breast carcinoma preferably is Estrogen Receptor (ER) positive, including ER and Progesterone Receptor (PR) positive.

The invention further provides a kit of parts, comprising a PARP inhibitor and an immune checkpoint inhibitor, as a combined preparation for separate or sequential use in the treatment of a HER2 negative, MammaPrint high risk 2 (MP2) breast carcinoma in a subject. Said kit of parts may further comprise a pharmaceutical composition comprising a suitable taxane.

For the purpose of clarity and a concise description, features are described herein as part of the same or separate aspects and preferred embodiments thereof, however, it will be appreciated that the scope of the invention may include embodiments having combinations of all or some of the features described.

The invention will now be illustrated by the following examples, which are provided by way of illustration and not of limitation and it will be understood that many variations in the methods described and the amounts indicated can be made without departing from the spirit of the invention and the scope of the appended claims.

TABLE 1
Overview of MammaPrint probes and signature genes.

| | Probe sequence | Gene | Ensemble ID | REF SEQ ID | Corr |
|---|---|---|---|---|---|
| 1 | CTGAGTGGTCAGAGATCTGTAAAGCATGACT | ALDH4A1 | ENSG00000159423 | NM_170726 | + |
| | TTCAAGGATGGTTCTTAGGGGACTGTGTA | | | | |
| 2 | AGGACTTGAATGAGGAAACCAACACTTTGAG | FGF18 | ENSG00000156427 | NM_003862 | + |
| | AAACCAAAGTCCTTTTTCCCAAAGGTTCT | | | | |
| 3 | GCCATTAAGATTTGGATGGGAAGTTATGGGT | CAPZB | ENSG00000077549 | NM_017765 | + |
| | AATGAGAATATAATGACATCTTGCAACAT | | | | |
| 4 | GATGGCCCAGCCTGTAAGATACTGTATATGC | BBC3 | ENSG00000105327 | NM_014417 | + |
| | GCTGCTGTAGATACCGGAATGAATTTTCT | | | | |
| 5 | GGCCTCACATTCTGCTCTGCTAAGTTTGGAG | EBF4 | ENSG00000088881 | XM_938882 | + |
| | AAAACAGAACAATAAACCAGATGCAGGTG | | | | |
| 6 | AAGTACTGGAATGTAATGGTTGAAATTCCTA | NA | NA | NT_022517 | + |
| | TTCAGTGATCTGGAAGAACTCTAATGTTC | | | | |
| 7 | CCAACGCACACCAGTCTTCTCAATCTGACTG | MYLIP | ENSG00000007944 | NM_013262 | + |
| | TAATCTAATCTGTTGTGCTTTTGTTGGAC | | | | |
| 8 | GGTTTAAAGCTGAAGAGGTTGAAGCTAAAAG | WISP1 | ENSG00000104415 | NM_003882 | + |
| | GAAAAGGTTGTTGTTAATGAATATCAGGC | | | | |
| 9 | GGCTAAAAGGGAAAAAGGATATGTGGAGAAT | GSTM3 | ENSG00000134202 | NM_000849 | + |
| | CATCAAGATATGAATTGAATCGCTGCGAT | | | | |
| 10 | CCTTTCAAACATGATCAAAGATTTCCCAATGT | RAB27B, | ENSG00000041353, | NM_004163 | + |
| | GATCTCATCATCATGGATACTCAATTTG | AC098848.1 | ENSG00000267112 | | |
| 11 | GGGGAACAATGAGGGCATTTCATGAACCATC | RTN4RL1 | ENSG00000185924 | NM_178568 | + |
| | TCAGGCACTTCTGCATCACGGAAGACCTG | | | | |
| 12 | TGCCTTGAGAATTTCAAAAGAGGTAATCAGG | ECI2 | ENSG00000198721 | NM_006117 | + |
| | AAAAGAGAGAGAGAAAAACTACACGCTGT | | | | |
| 13 | GTCTGGGATTAAGGGCAAATCTATTACTTTT | TGFB3 | ENSG00000119699 | NM_003239 | + |
| | GCAAACTGTCCTCTACATCAATTAACATC | | | | |
| 14 | TAAAAAAGAAATAGTCAGTGTTTTCCTCCTTT | STK32B | ENSG00000152953 | NM_018401 | + |
| | CAACCGAGACTATTTCTGGATTGTGTGC | | | | |
| 15 | TTTTCAGAAAGAAGTCTGGACCAGGCTGAAG | ECI2 | ENSG00000198721 | NM_206836 | + |
| | GCATTTGCAAAGCTTCCCCCAAATGTCTT | | | | |
| 16 | CCTCATTGCCTTATTCGGAGTACTATTATCCA | MS4A7, MS4A14 | ENSG00000166927; | NM_206939 | + |
| | ATATATGAAATCAAAGATTGTCTCCTGA | | ENSG00000110079 | | |
| 17 | TGGCATCATACAAAGAGCAGGAGAAGCAAAC | AP2B1 | ENSG00000006125 | NM_1030006 | + |
| | ACCCAGAACTCTTTTGCTGGTCAGAGATT | | | | |
| 18 | TCCAGACCTACCTTGTACGCACATAGACATTT | DHX58 | ENSG00000108771 | NM_024119 | + |
| | TCATATGCACTGGATGGAGTTAGGGAAA | | | | |
| 19 | ATCTTTGTTAATTATTTTGGGGAGTAGTTGGG | RAI2 | ENSG00000131831 | NM_021785 | + |
| | AAATGGAAAGGTGAATTGGCTCTAGAGG | | | | |
| 20 | GTTCATTTCCAGCCCTTTCTAGATCTGATCTT | HIPK2 | ENSG00000064393 | NT_007933 | + |
| | TTAGGGGGAAAGACAGCTTAAAATGTTC | | | | |
| 21 | TGAATGTCATGTTTATGTCATAGACGTAGAA | QDPR | ENSG00000151552 | NM_000320 | + |
| | AACGCATCCTTGAATTAAACTGCCTTAAC | | | | |
| 22 | TACTGGAGTAACTGAGTCGGGACGCTGAATC | ZG16B | ENSG00000162078 | NM_145252 | + |
| | TGAATCCACCAATAAATAAAGCTTCTGCA | | | | |
| 23 | CAGATTCCCCAGAAACTACCTTTTGCCCAAA | NEO1 | ENSG00000067141 | NM_002499 | + |
| | GAACATGCTCAGTATTTGGGGCATTTCCT | | | | |
| 24 | AGGCAGGGGTGGTGATTCATGCTGTGTGACT | ACADS | ENSG00000122971 | NM_000017 | + |
| | GACTGTGGGTAATAAACACACCTGTCCCC | | | | |
| 25 | TGGATTTCTAAACTGCTCAATTTTGACTCAAA | BTG2 | ENSG00000159388 | NM_006763 | + |
| | GGTGCTATTTACCAAACACTCTCCCTAC | | | | |
| 26 | CCAATCCAACAACTATAGGCTGGGTTAAATA | BBOF1, ALDH6A1 | ENSG00000119636, | NM_005589 | + |
| | AAAGGTCATTATTGTCTATATTCCAAGTG | | ENSG00000119711 | | |
| 27 | TCTACCACATTAAATTCTCCATTACATCTCAC | LYPD6, | ENSG0000018712 | NM_194317 | + |
| | TATTGGTAATGGCTTAAGTGTAAAGAGC | LINC00474 | ENSG00000204148 | | |
| 28 | TGAGGAATTCTTGTACGCAGTTTTCTTTGGCT | CIRBP | ENSG00000099622 | NM_001280 | + |
| | TTACGAGCCGATTAAAAGACCGTGTGAA | | | | |
| 29 | CTGGTCTTTGAAAGAAATGTACTACTAAAGA | AC07914, MATN3 | ENSG00000227210E | NM_002381 | + |
| | GCACTAGTTGTGAATTTAGGGTGTTAAAC | | NSG00000132031 | | |
| 30 | ATGATGGGAGAGCTCTGGCAGATGTCCCAAT | INPP5J | ENSG00000185133 | NM_014422 | + |
| | CCTGGAGGTCATCCATTAGGAATTAAATT | | | | |
| 31 | CAACTTGCTCTTTCATATGAGTTGGTCATAGC | FGD6 | ENSG00000180263 | NT_019546 | + |
| | ATGTAAGAACCAATCTTGAAATATCGTT | | | | |
| 32 | CCTGGATCAGAGTAAGAATGTCTTAAGAAGA | CACNA1D, CHDH | ENSG00000157388, | NT_022517 | |
| | GGTTTGTAAGGTCTTCATAACAAAGTGGT | | ENSG00000016391 | | |
| 33 | CTCCTGGACTGCTTCTTTTGGCTCTCCGACAA | SDSL | ENSG00000139410 | NM_138432 | |
| | CTCCGGCCAATAAACACTTTCTGAATTG | | | | |
| 34 | AACCAACCCATAATTGCATTTTACTTGTCGTG | MINOS1, NBL1, | ENSG00000173436, | NM_001032363 | |
| | GTTCGATCTGATTGTATTGTCGAAGGAC | MINOS1-NBL1 | ENSG00000158747, | | |
| | | | ENSG00000270136 | | |
| 35 | ATTCCTTTATGAGCTCTCCATATCCTTCTTGA | PEX12 | ENSG00000108733 | NM_000286 | + |
| | GAAACTGGTTAAAAAAGGAATAGGGGTA | | | | |
| 36 | AGTGGGGGTTGTGTAAAGGGGAAGTCATCTT | ERGIC1 | ENSG00000113719 | NM_001031711 | + |
| | TTGAGATCCAGATAGACATGGTTTGTGCA | | | | |
| 37 | TCAGCTTAAGTACTTATTGTGGTAGTGAGTC | FBXO16 | ENSG00000214050 | NM_172366 | + |
| | CTACGGTATTTCAGTAAAAAGGAATTCAT | | | | |
| 38 | GGCAAGAGTTATCATAGAACAACAAAATAGA | ZNF385B | ENSG00000144331 | NM_152520 | + |
| | GTGGACTCTTTTAGAGCATCTATATCTGC | | | | |
| 39 | GGAGTTTCTGTTTAGGGCATTAAAAATTCCC | IP6K2 | ENSG00000068745 | NM_1005913 | + |
| | GCAAACTATAAAGAGCAATGTTTTCAGTC | | | | |
| 40 | ATAATTCTCTGTACAGGGGGGTTTGTGCTAT | MARCH8 | ENSG00000165406 | NM_145021 | + |
| | ACACTGGGATGTCTAATTGCAGCAATAAA | | | | |
| 41 | AGGACTTTAATCTTGGTGATGCCTTGGACAG | CMTM8, | ENSG00000170293, | NM_199187 | + |
| | CAGCAACTCCATGCAAACCATCCAAAAGA | KRT18P15, | ENSG00000234737, | | |
| | | KRT18P34, | ENSG00000244515, | | |
| | | KRT18P13, | ENSG00000214417, | | |
| | | PCDH11Y, | ENSG00000099715, | | |
| | | KRT18P10 | ENSG00000214207 | | |
| 42 | GGGCAAAATGTATCACTCCAAACACTACTGA | RUNDC1 | ENSG00000198863 | NM_173079 | + |
| | TTCAGCATTGTTTTCATGTCTTAAAATTG | | | | |
| 43 | CTGGATGTTTAGCTTCTTACTGCAAAAACATA | TBC1D9 | ENSG00000109436 | NM_015130 | + |
| | AGTAAAACAGTCAACTTTACCATTTCCG | | | | |
| 44 | GGTAACTTGCAGGAATATTCTATTGGAAAAG | LETMD1 | ENSG00000050426 | NM_015416 | + |
| | ATAACAGGAAGTACAAGTGCTTCTTGACC | | | | |
| 45 | TCAATGGTTAGCAGAAGGGAGAAAAGAAAGC | RILPL2 | ENSG00000150977 | NP_659495 | + |
| | AGGAAAATGTGCTATTGAGATTCCAGTGG | | | | |
| 46 | CCTGGGTTTACAACGCTGTTAGGAAAATTAA | SEC14L2, | ENSG00000100003, | NM_012429 | + |
| | CCAATGAATAAAGCAACGTTCAGTGCGCA | AC004832.3 | ENSG00000249590 | | |
| 47 | TTTTTGTACCTTGTCACTATAACTACTTCCTA | KIAA1217 | ENSG00000120549 | NM_019590 | + |
| | GTCAAAGAACGAAATGTAACTGTTACCG | | | | |
| 48 | TTCTAGCTGTTATTTTGCTATTTGGCATTTAC | CCDC74A, | ENSG00000163040, | NM_207310 | + |
| | ATAAAAGCACACGATGAAGCAGGTATCG | MED15P9, | ENSG00000223760, | | |
| | | CCDC74B | ENSG00000152076 | | |
| 49 | TTGGGTTTATTTCCAGGTCACAGAATTGCTGT | TBX3 | ENSG00000135111 | NM_005996 | + |
| | TAACACTAGAAAACACACTTCCTGCACC | | | | |
| 50 | GAACAGCTCCTTACTCTGAGGAAGTTGATTC | FUT8 | ENSG00000033170 | NM_178157 | + |
| | TTATTTGATGGTGGTATTGTGACCACTGA | | | | |
| 51 | CTTTCTTATTTACTAAGAATTTGCCTGTTTGA | KIF3B | ENSG00000101350 | NM_004798 | + |
| | ATAAGAACAAAACGCTAAGGTGGGTAGC | | | | |
| 52 | CTAGAGAGCAGAAATAAAAAGCATGACTATT | PCAT7, FBP1 | ENSG00000231806, | NM_000507 | + |
| | TCCACCATCAAATGCTGTAGAATGCTTGG | | ENSG00000165140 | | |
| 53 | GTTCAGGGGCATCACCTACTTTGCTTACTTG | LBHD1, CSKMT | ENSG00000162194, | NM_024099 | + |
| | ATTCAAGGCTCTCATTAAAGACATTTTAG | | ENSG00000214756 | | |
| 54 | GTTGGTAGAGGGAGTATGATAAAATGTTTAA | KIAA1324 | ENSG00000116299 | NM_020775 | + |
| | ATCTCATTTGGTTACCTTGAGTCCTGGAA | | | | |
| 55 | AATTCAACAGTGTGGAAGCTTTAGGGGAACA | TMEM25 | ENSG00000149582 | NM_032780 | + |
| | TGGAGAAAGAAGGAGACCACATACCCCAA | | | | |
| 56 | CAAGTTGTGCAAAGTGAGAAAGATCTTTGTG | PIN4, RPS4X | ENSG00000102309, | NM_001007 | + |
| | GGCACAAAAGGAATCCCTCATCTGGTGAC | | ENSG00000198034 | | |
| 57 | CAAGAGAACCTGGAGAAAACTACCGTATTCA | STON2 | ENSG00000140022 | NT_026437 | + |
| | AGAGATTAATCAAAATCAGTGTTTTAGCC | | | | |
| 58 | CCGAATGACCTTAAAGGTGATCGGCTTTAAC | TENM3 | ENSG00000218336 | XM_940722 | + |
| | GAATATGTTTACATATGCATAGCGCTGCA | | | | |
| 59 | AGTTTATGGGCCAGAATATTCTGTATACCAG | RASL11B | ENSG00000128045 | NM_023940 | + |
| | ACATTGGTAAGCTCTCATGGTTTACAGGA | | | | |
| 60 | CCATGTGGCCAGTCTACCATGGGGCCCAGGA | GSDMD | ENSG00000278718 | NM_024736 | + |
| | GTTGGGGAAACACAATAAAGGTGGCATAC | | | | |
| 61 | ATGCTTAAACCCACGGAAGGGGGAGACTCTT | LAMP5 | ENSG00000125869 | NM_012261 | + |
| | TCGGATTTGTAGGGTGAAATGGCAATTAT | | | | |
| 62 | TTCTTTCTTCAAAGAGTCATCAGAATAACATG | CHPT1, SYCP3 | ENSG00000111666, | NM_153694 | + |
| | GATTGAAGAGACTTCCGAACACTTGCTA | | ENSG00000139351 | | |
| 63 | TGAAGTCAGCGTTAACCATGTGCATACAACT | ZNF627 | ENSG00000198551 | NM_145295 | + |
| | TAAGGAATTTTTTCCTCCTCATGTAAATT | | | | |
| 64 | GTTAAACAGGGATTATAGTACTTGTCTCACA | COL23A1 | ENSG00000050767 | NT_023133 | + |
| | AAGTTTCTGTGAGAATTAAACAAGGGGAT | | | | |
| 65 | CAGCCTGTGTGATACAAGTTTGATCCCAGGA | SCUBE2 | ENSG00000175356 | NM_020974 | + |
| | ACTTGAGTTCTAAGCAGTGCTCGTGAAAA | | | | |
| 66 | AATGCACAGATCTGCTTGATCAATTCCCTTGA | AC023024.1, | ENSG00000259172, | NM_138319 | + |
| | ATAGGGAAGTAACATTTGCCTTAAATTT | PCSK6 | ENSG00000140479 | | |
| 67 | TTTCCAATAACCACCTAAATTTTAACAAAGGT | RBP3 | ENSG00000265203 | NM_002900 | + |
| | TCCTTCTAAGTGGTAGAACTTGGGGTGG | | | | |
| 68 | AGTTATGCTTCCCTTCATGTTATATGCACATT | MYRIP, | ENSG00000170011, | NM_015460 | + |
| | GCCAAGAATTACTGTCAAGAGAAATGAT | EIF1B-AS1 | ENSG00000280739 | | |
| 69 | AAGGTTTGAAGGTTACGGCTCAGGGCTGCCC | SPEF1 | ENSG00000101222 | NM_015417 | + |
| | CATTAAAGTCAGTGTTGTGTTCTAAAAAA | | | | |
| 70 | GGACTGTATGAATTTATAGAAAATTGAATCTA | CLSTN2 | ENSG00000158258 | NT_005612 | + |
| | ATTTCAGAAGAGCGCACTGTCTTCTCAG | | | | |
| 71 | TACATTTCTTTGGGTTTCTAGAGACGCCCCTA | EVL, DEGS2 | ENSG00000196405, | NM_016337 | + |
| | AGTCACCTGCTTCATTAGACGGTTTCCA | | ENSG00000168350 | | |
| 72 | GGCCTAATTGAGGGAAGGAGGAAATTCATAC | ELMOD3 | ENSG00000115459 | NM_032213 | + |
| | CAGCAGTTTTCAAATAAAAGAATTGTTCT | | | | |
| 73 | TCCAATTCTACACTCAGTTAAAGACCATTACT | BBOF1, ALDH6A1 | ENSG00000119636, | NM_005589 | + |
| | TCTCAGTGGAAAGAAGAAGATGCTACTC | | ENSG00000119711 | | |
| 74 | GTGGGGACTTCGTGGGAGGCACTCATGGCTC | KIAA1683 | ENSG00000130518 | NM_025249 | + |
| | TCTGGGTCTAATGAATAAAGTCCTCCACA | | | | |
| 75 | CCAGGATCTTAAGGAAGAATATTCTAGGAAG | SPC25 | ENSG00000152253 | NM_020675 | − |
| | AAGGAAACTATTTCTACTGCTAATAAAGC | | | | |
| 76 | AGAAAACCCTTTTCTACAGTTAGGGTTGAGT | TFRC | ENSG00000072274 | NM_003234 | − |
| | TACTTCCTATCAAGCCAGTACGTGCTAAC | | | | |
| 77 | TAGGGAATGAATGAATGAATATGGATTGCTG | PAQR3 | ENSG00000163291 | NM_177453 | − |
| | TTAACTAGAAACACTTCTGTATGTCAGTC | | | | |
| 78 | GTACTTAGCTGGAAGAACATGTTAATTCTGC | MLLT10 | ENSG00000078403 | NM_1009569 | − |
| | AATATGTTTCTTGGTTAAACATTGCACAG | | | | |
| 79 | ACTCTCTTAGGTCATTTTTCAATGTGTGTAAC | CENPBD1 | ENSG00000177946 | NM_145039 | − |
| | CAAAAGTTAATCAGAATAAAGCGGAAGC | | | | |
| 80 | AATGCTTTGTTGGAGTTTAAAAATTCAGGGA | AL44926, GPSM2, | ENSG00000274068, | NM_013296 | − |
| | AAAAATCGGCAGACCATTAGTTACTATGG | CLCC1 | ENSG00000121957, | | |
| | | | ENSG00000121940 | | |
| 81 | AAGAAACCAGCATGTGACTTTCCTAGATAAC | PIMREG | ENSG00000129195 | NM_019013 | − |
| | ACTGCTTTCTCATAATAAAGACTATTTGC | | | | |
| 82 | GTTGGCATTGATATGGTACAACCTGCAAATT | HACD2 | ENSG00000206527 | NM_198402 | − |
| | ACTTGCAGTTCTGAGTTTCAGATAAAACA | | | | |
| 83 | AGTGTCATTTTAAGGGACATTTTTATGACTTT | ACE, | ENSG00000159640, | NM_152831 | − |
| | TATGTGTATGTTTATGTAGAAATTTGGA | AC113554 | ENSG00000264813 | | |
| 84 | ACTCACTTCTTTTCAGGTGTAGCTACAATTGT | OXCT1 | ENSG00000083720 | NM_000436 | − |
| | GTAATGTACAATATTAGAGAAAGGACAG | | | | |
| 85 | CCTGGGAGCAAATGAACAATAGCTAAGTGTC | GNAZ | ENSG00000128266 | NM_002073 | − |
| | TTGGTATTTAAAGAGTAAATTATTTGTGG | | | | |
| 86 | CCAAGAATATATGCTACAGATATAAGACAGA | FLT1 | ENSG00000102755 | NM_002019 | − |
| | CATGGTTTGGTCCTATATTTCTAGTCATG | | | | |
| 87 | ATGCTTTCCTAAATCAGATGTTTTGGTCAAGT | MAD2L1, MNAT1 | ENSG00000164109, | NM_002358 | − |
| | AGTTTGACTCAGTATAGGTAGGGAGATA | | ENSG00000020426 | | |
| 88 | ATTTGTGTGGACAAAAATATTTACACTTAGG | CDC25B | ENSG00000101224 | NM_004358 | − |
| | GTTTGGAGCTATTCAAGAGGAAATGTCAC | | | | |
| 89 | AAATATACTATGTTTGCGAACCTTGGTAGCTA | KIF21A | ENSG00000139116 | NM_017641 | − |
| | TGATGAGAGCTATTATCATCTGTGGTGG | | | | |
| 90 | TCAATGAAAGTTCAAGAACCTCCTGTACTTAA | HMGB3 | ENSG00000029993 | NM_005342 | − |
| | ACACGATTCGCAACGTTCTGTTATTTTT | | | | |
| 91 | ACCTTGATAGTTCACCACGTCTGATGGATCC | PTDSS1 | ENSG00000156471 | NM_014754 | − |
| | CTGTTTTAAATAAAAACGATTCACTTTAA | | | | |
| 92 | TAAAATACTTCAATCCTGGATTCACAGTGGG | MTMR2 | ENSG00000087053 | NM_016156 | − |
| | AACAAGTTTCTATTAAAAGGCAAATGCTG | | | | |
| 93 | GGCTGTGAACAATGTTAAATAGCATCAGTTT | CENPU | ENSG00000151725 | NM_024629 | − |
| | GTCCAATAGTTTTAAAGGCCATAATCATC | | | | |
| 94 | ACGAGTACCGGCATGTTATGTTACCCAGAGA | AL353705 | ENSG00000234819 | NM_001827 | − |
| | ACTTTCCAAACAAGTACCTAAAACTCATC | | | | |
| 95 | ATTTTTTAGAAAATACACACTTTTCAGGAGAA | Clorf198 | ENSG00000119280 | NM_032800 | − |
| | ACCTGAGCATGATTTTGGATTCTCCACC | | | | |
| 96 | CAGCTCAGACCATTTCCTAATCAGTTGAAAG | RRM2, | ENSG00000171848, | NM_001034 | − |
| | GGAAACAAGTATTTCAGTCTCAAAATTGA | AC007240 | ENSG00000284681 | | |
| 97 | CACTGCAGACTCTCAAGAGATCAATCAAATT | INTS7 | ENSG00000143493 | NM_015434 | − |
| | GCCAGAAACAGTTTGGTTTTTCATATGGA | | | | |
| 98 | TGAAACTTTCTTCTGATGAGTTTCTTTAACGT | MRPL13 | ENSG00000172172 | NM_014078 | − |
| | ACAGGATGGAGTAAAACAAATGGTACAG | | | | |
| 99 | CAATTCTTGAGAGTTAATGTGATCATGATATT | ARMC1 | ENSG00000104442 | NM_018120 | − |
| | GCAAACAACTATAAATGGTCTCTAGGCC | | | | |
| 100 | GAAGGAAACACCGAGTCTCTGTATAATCTAT | ADM | ENSG00000148926 | NM_001124 | − |
| | TTACATAAAATGGGTGATATGCGAACAGC | | | | |
| 101 | AGCAACCTGGGCCTTGTACTGTCTGTGTTTTT | IGFBP5 | ENSG00000115461 | NM_000599 | − |
| | AAAACCACTAAAGTGCAAGAATTACATT | | | | |
| 102 | GGGAATTTGATGCAGTAAAGATTACCCTGTT | SKA3 | ENSG00000165480 | NM_145061 | − |
| | TTATGATTGTTCCTTGAAAGTCAAATGGG | | | | |
| 103 | TAAGGCTAATGATACCAATGAGGGTTGGTTT | SLC7A1 | ENSG00000139514 | NM_003045 | − |
| | ATTATCAAACCTGAATAGCTGTGGTTTCT | | | | |
| 104 | TGGGGAGATACATCTTATAGAGTTAGAAATA | PRAME | ENSG00000185686 | NM_006115 | − |
| | GAATCTGAATTTCTAAAGGGAGATTCTGG | | | | |
| 105 | TATCTTGAAACTGACCAAACGCTTATTGTGTA | CTSV | ENSG00000136943 | NM_001333 | − |
| | AGATAAACCAGTTGAATCATTGAGGATC | | | | |
| 106 | TTCTCTGAAGGAATCATGTTCAGTGTTCGAC | SMC4P1 | ENSG00000229568, | NM_1002799 | − |
| | CACCTAAGAAAAGTTGGAAAAAGATCTTC | AC07959 | ENSG00000248710, | | |
| | | SMC4 | ENSG00000113810, | | |
| | | TRIM59 | ENSG00000213186 | | |
| 107 | TGTCATAGACATGTATTGGGGAGCTTCCAAT | NIPA1 | ENSG00000170113 | NM_144599 | − |
| | TAGCATACATAGACACATGTGTCAGTGGC | | | | |
| 108 | TGTCCATGCTACAAGAAGTTATGAGCCTTGT | SFT2D2 | ENSG00000213064 | NM_005149 | − |
| | TCTAAGTACAGATGAACCTTGTATTTGTG | | | | |
| 109 | ATCCCGATTTCAGTCAGACAAATACTCATTTC | SACS | ENSG00000151835 | NM_014363 | − |
| | AGAGATTCTATACTTCATGGAATCAAGA | | | | |
| 110 | AGTTACTTTCTTAATGTGACCTAGCAATAGGC | CTPS1 | ENSG00000171793 | NM_001905 | − |
| | ATAGCTACGTGGCACTATATTCTGGCCA | | | | |
| 111 | GAAATCTCTCTACACAGATGAGTCATCCAAA | NUSAP1 | ENSG00000137804 | NM_018454 | − |
| | CCTGGGAAAAAATAAAAGAACTGCAATCA | | | | |
| 112 | AAATTGCTAAGTGGAATGCATGAATTGCATT | PSMD7 | ENSG00000103035, | NM_002811 | − |
| | ATGTTCTCTGGTAACACGTAGAGTTCAGA | AC009120 | ENSG00000259972 | | |
| 113 | CCAAAGGTCTTGGTACAACCAGCTGCCCATT | BUD23 | ENSG00000071462, | NM_004603 | − |
| | TTGTGAAATTTTTATGTAGAATAAACATT | STX1A | ENSG00000106089 | | |
| 114 | GTTTCGGGTCTTTACCTCATAGTATGAAATTA | KIAA1147 | ENSG00000257093 | XM_1130020 | − |
| | GTAAGACACTGCATAGATTTTGCCCTGA | | | | |
| 115 | GAGTACGGATGGGAAACTATTGTGCACAAGT | NDRG1 | ENSG00000104419 | NM_006096 | − |
| | CTTTCCAGAGGAGTTTCTTAATGAGATAT | | | | |
| 116 | TATTTTATCAGCACTTTATGCACGTATTATTG | PFKP | ENSG00000067057, | NM_002627 | − |
| | ACATTAATACCTAATCGGCGAGTGCCCA | AL45116 | ENSG00000278419 | | |
| 117 | TGCCCTATGGAAAACTTGTCCAAATAACATTT | CD163L1 | ENSG00000177675 | NM_174941 | − |
| | CTTGAACAATAGGAGAACAGCTAAATTG | | | | |
| 118 | CTCCTTGTCATTGACCTTAGCTAAACCATGGC | MAPRE2 | ENSG00000166974 | NM_014268 | − |
| | AATTCATAAATAGAGGAAACATTAATGA | | | | |
| 119 | CTGAACGAGAACAAGAATCAGAAGAAGAAAT | TMEM45A | ENSG00000181458 | NM_018004 | − |
| | GTGACTTTGATGAGCTTCCAGTTTTTCTA | | | | |
| 120 | TATATTATCAGTCTGTACCAGTAGACCAGTAC | PABPC1 | ENSG00000070756 | NM_002568 | − |
| | CCTAACTACTGAAAAGAATATGGCAGTT | | | | |
| 121 | AGTAACGCTAACTTTGTACGGACGATGTCTC | RHBDF2 | ENSG00000129667 | NM_001005498 | − |
| | ATGGATTAAATAATATTCTTTATGGCAGT | | | | |
| 122 | GTGGATCTACCTCAGTTAAACAGTTGGGTGC | AGO2 | ENSG00000123908 | AF093097 | − |
| | TATTACTAAGTCTGTCAAATTAAATTGGA | | | | |
| 123 | CATTCTAAAGGGAAATCAGTAAAATGTCTTG | TMEM64 | ENSG00000180694 | NM_1008495 | − |
| | ATAATTGGTATCCAAATCACTTGTGTGCC | | | | |
| 124 | CCAAAGACAAACGATTAGAAGATGGCTATTT | MGAT4A | ENSG00000071073 | NM_012214 | − |
| | CAGAATAGGAAAATTTGAGAATGGTGTTG | | | | |
| 125 | CAAACTTCCTGACACTACTTCCATATTTGCAC | CDK16 | ENSG00000102225 | NM_006201 | − |
| | TAAAGGAGATTCAGCTACAAAAGGAGGC | | | | |
| 126 | ACCTTCCTATGAAGATCATGGAATCAAATAC | AL589666 | ENSG00000271793, | NM_006372 | − |
| | GGGACATTGAACTAATACTTGGACTTTGA | SYNCRIP | ENSG00000135316 | | |
| 127 | GGCTAACACAATCTAATTTTGGTTTAAGAGA | HIF1A | ENSG00000100644 | NM_181054 | − |
| | CAAATCTAGAGTCTCAAATGATCTCAGAG | | | | |
| 128 | TGGACCCTTAAATATGACTAAAATCACAGCA | RRAGD | ENSG00000025039 | NM_021244 | − |
| | ATATTGTTACATACGGGTTATATGCCAAC | | | | |
| 129 | TAAGCATTGTGAAGGAAGATTAATATAGCCA | HIF1A | ENSG00000100644 | NM_181054 | − |
| | AATAACTAGAGTGATCAGTTCTACCAGAG | | | | |
| 130 | CCTGGATAAAAGTACTGTATGATTTTGTGAT | DEGS1 | ENSG00000143753 | NM_144780 | − |
| | GGATGATACAATAAGTCCCTACTCAAGAA | | | | |
| 131 | GCTTTGTTACTTTGTTAGGTACGAATCACATA | LRP12 | ENSG00000147650 | NM_013437 | − |
| | AGGGAGATTGTATACAAGTTGGAGCAAT | | | | |
| 132 | TAAAAGATGAAGAAAGCTATTAGGTATATTT | ZDHHC20 | ENSG00000180776 | NT_024524 | − |
| | GTACATGACTGCAAATGAGTCTATGCCCG | | | | |
| 133 | GTGTGTTATCTTTATATGTCAAACTGGTTGAA | PLEKHA1 | ENSG00000107679 | NM_021622 | − |
| | CACTGTAATGAGAATAAACTGCACAGAG | | | | |
| 134 | GATTATTGTACGAAGTGTCTCTGTAATTATCA | FBX05 | ENSG00000112029 | NM_012177 | − |
| | TACTACTAAAGACTGTTCAGATGGCAAG | | | | |
| 135 | CATTTGTATTAATGGAATACTAAGTCCCTCTG | NEAT1 | ENSG00000245532 | NT_033903 | − |
| | TGATTTCTGAACCAAGCTATTCCTAGGC | | | | |
| 136 | ATGAAGAGATTTCTCAAGCTATTCTTGATTTC | PIR | ENSG00000087842 | NM_003662 | − |
| | AGAAACGCAAAAAATGGGTTTGAAAGGG | | | | |
| 137 | AGCCAATCATGAGTACGTAAAGTGATTTTTG | ASPM | ENSG00000066279 | NM_018136 | − |
| | CTCTCTGTGTACAACTTTTAAAATCTGAC | | | | |
| 138 | ATCCTAGACCATATTTTCAAGTCATCTTAGCA | GBE1 | ENSG00000114480 | NM_000158 | − |
| | GCTAGGATTCTCAAATGGAAGTGTTATA | | | | |
| 139 | AGTGATTTCATGCTAGAAAAATTGGAAACTA | HJURP | ENSG00000123485 | NM_018410 | − |
| | AAAGTGTGTAGCTAGGTTATTTCGGAGTG | | | | |
| 140 | GCTAAGCCAAGTAGTAGCAGTAAAACTTCTG | QSER1 | ENSG00000060749 | NM_024774 | − |
| | ATCCTCTAGCATCAAAAACTACAACTACA | | | | |
| 141 | GGAAAGAAGTTGAAAGCATCTTGAAGAAAAA | BNIP3 | ENSG00000176171 | NM_004052 | − |
| | CTCAGATTGGATATGGGATTGGTCAAGTC | | | | |
| 142 | ACCTGGATATGTCTGTGAGGCTCCTGAAAGG | AC087521 | ENSG00000254409, | BC052560 | − |
| | AGACAAATAAAGTCAATATATTTGCACAA | C11orf96 | ENSG00000187479, | | |
| | | AC087521 | ENSG00000244953 | | |
| 143 | GGGTATGAAAGATGAGTGTCTGTAAAAATCC | LINC00888 | ENSG00000240024 | NT_005612 | − |
| | TTCTTAGAAATGTATTTCCTCAAGACTCT | | | | |
| 144 | CAGATGGCAAGATTGAGTTTATTTCAACAAT | GGH | ENSG00000137563 | NM_003878 | − |
| | GGAAGGATATAAGTATCCAGTATATGGTG | | | | |
| 145 | GAAACTGTGTCACCCTAAAGAAGCATATAAT | TRIP13 | ENSG00000071539 | NM_004237 | − |
| | CATAGCATTAAAAATGCACACATTACTCC | | | | |
| 146 | CAAGCGTGTTTCTAGAGAACAGTTGAGAGAG | STMN1 | ENSG00000117632 | NM_005563 | − |
| | AATCTCAAGATTCTACTTGGTGGTTTGCT | | | | |
| 147 | CCGACAAGAGGAGATCATTTTAGATATTACC | CENPN | ENSG00000166451, | NM_018455 | − |
| | GAAATGAAGAAAGCTTGCAATTAGTGAAC | AC092718 | ENSG00000260213, | | |
| | | AC092718 | ENSG00000284512 | | |
| 148 | TAATAGCAAAATTTAACCCGTTACTCTTTAAC | MYO10 | ENSG00000145555 | NM_012334 | − |
| | CTTGTACTGGAAATTCTAAGCAGTGCAG | | | | |
| 149 | CTTCCTACCTCTGGTGATGGTTTCCACAGGA | TK1 | ENSG00000167900 | NM_003258 | − |
| | ACAACAGCATCTTTCACCAAGATGGGTGG | | | | |
| 150 | AAATCATTCGGTAAATCCAAACTGCTATGCA | RUNX1 | ENSG00000159216, | NM_004456 | − |
| | AAAGTTATGATGGTTAACGGTGATCACAG | EZH2P1 | ENSG00000231300 | | |
| 151 | TTGGGTTTCTAGTCCTCCTTACCATCATCTCC | AURKA | ENSG00000087586 | NM_003600 | − |
| | ATATGAGAGTGTGAAAATAGGAACACGT | | | | |
| 152 | GCTGGTGGAGTAGCAGATGATATTAATACTA | DLGAP5 | ENSG00000126787 | NM_014750 | − |
| | ACAAAAAAGAAGGAATTTCAGATGTTGTG | | | | |
| 153 | TCACCCAGAACCAATGCGGTGTTTCTTAATG | TBCE | ENSG00000285053, | NM_152490 | − |
| | TTTGCACAAATTTCCTTAAAAATCAACTT | B3GALNT2 | ENSG00000162885 | | |
| 154 | CAGGACTTCTCTTTAGTCAGGGCATGCTTTAT | CENPF | ENSG00000117724 | NM_016343 | − |
| | TAGTGAGGAGAAAACAATTCCTTAGAAG | | | | |
| 155 | CCCTGTGCTATCGTAAGTTTGTTTTGAGCACT | AL117350 | ENSG00000237481, | NM_145257 | − |
| | GCATTCACTTTAAAATTCTGGAGGAACA | CCSAP | ENSG00000154429 | | |
| 156 | CAACATATTTCAGTTGGAAAATTTGTATGCAG | ATAD2 | ENSG00000156802 | NM_014109 | − |
| | TAATCAGCCAATGTATTTATCGGCATCG | | | | |
| 157 | CCCCCATTCTGGAAGGTTTTGTTATCTTCGGA | PSMD2 | ENSG00000175166, | NM_002808 | − |
| | AGAACCCCAATTATGATCTCTAAGTGAC | FMN2 | ENSG00000155816, | | |
| | | AL359918 | ENSG00000228818 | | |
| 158 | TGTCCCCAGGGATCAAACAGAAGCAGCCGTG | SHMT2 | ENSG00000182199, | NM_020142 | − |
| | GGCAAAATACAATTTCATTTAACAAATTG | NDUFA4L2 | ENSG00000185633 | | |
| 159 | AAACAGCATTATGGAGTTAAAAGATTTTTACA | PIMREG | ENSG00000129195, | NM_019013 | − |
| | ACTGGGTCTTGATTTTGATGTGAGCTGG | PITPNM3 | ENSG00000091622 | | |
| 160 | TCCAGACGCACTGATCTTTGCAAAGGAGACT | DCK | ENSG00000156136 | NM_000788 | − |
| | TAATTTCAAATCTGTAATTACCATACATA | | | | |
| 161 | CATTTGGCTGTCAGAAATTATACCGAGTCTA | DTL | ENSG00000143476 | NM_016448 | − |
| | CTGGGTATAACATGTCTCACTTGGAAAGC | | | | |
| 162 | TTAAAGGCAAAACTGTGCTCTTTATTTTAAAA | COL4A2 | ENSG00000134871 | NM_001846 | − |
| | AACACTGATAATCACACTGCGGTAGGTC | | | | |
| 163 | AAGGTGCTGTCATATATCTTGGAATGAATGA | AGFG1 | ENSG00000173744 | NM_004504 | − |
| | CCTAAAATCATTTTAACCATTGCTACTGG | | | | |
| 164 | GGATGTAAATCCTGAGCTCAAATCTCTGTTA | NMB | ENSG00000197696 | NM 205858 | − |
| | CTCCATTACTGTGATTTCTGGCTGGGTCA | | | | |
| 165 | CCTCAAGAGTATGTATAATTTGAAGAGATAC | KIF14 | ENSG00000118193 | NM_014875 | − |
| | TTTGTAACTATGCTTGGGTGATATTGAGC | | | | |
| 166 | TTCACAGAATAGCACAAACTACAATTAAAACT | BIRC5 | ENSG00000089685 | NM_001012271 | − |
| | AAGCACAAAGCCATTCTAAGTCATTGGG | | | | |
| 167 | CCAGCACATAGGAGAGATGAGCTTCCTACAG | VEGFA | ENSG00000112715 | NM_003376 | − |
| | CACAACAAATGTGAATGCAGACCAAAGAA | | | | |
| 168 | GAGAAACATTGTATATTTTGCAAAAACAAGA | ECT2 | ENSG00000114346 | NM_018098 | − |
| | TGTTTGTAGCTGTTTCAGAGAGAGTACGG | | | | |
| 169 | TACTTTTTGGAAAAGAATAAACCAAGAATTG | IVNS1ABP | ENSG00000116679 | NM_016389 | − |
| | ATTGGGCACATCATTTCAAGAAGTCCCTC | | | | |
| 170 | ATGGAGTTGCTAGTAAAGCGAAGCTGATTAT | MCCC1 | ENSG00000078070 | NM_020166 | − |
| | CCTGGAAAACACTATTTACCTATTTTCCA | | | | |
| 171 | GACTGCTAGTGGATAATAACATCTTGACTAC | TMEM38B | ENSG00000095209, | NM_017779 | − |
| | TTAAAAAAGGGACATATTGAAAATCCTGG | AL592437 | ENSG00000232486, | | |
| | | OTUD7A | ENSG00000169918, | | |
| | | AC026951 | ENSG00000259358, | | |
| | | DEPDC1 | ENSG00000024526, | | |
| | | AL138789 | ENSG00000233589 | | |
| 172 | CATGTTACCTGGACTGGAACAGACTGTGAAT | INAVA | ENSG00000163362, | NM_018265 | − |
| | ATAGCAGAAGGTTCCAAGAACTCTGGTGT | SLC9C1 | ENSG00000172139 | | |
| 173 | GAGACCAGGTGCTTCAAAACTTAGGCTCGGT | KIF21A | ENSG00000139116 | NM_017641 | − |
| | AGAATCTTACTCAGAAGAAAAAGCAAAAA | | | | |
| 174 | GGATTCAACCCAAATGATTTCTCATCAGGTG | C16orf95 | ENSG00000260456 | AK026130 | − |
| | ATTCTTGGTTGTAGCAAAGTTCATGTGAA | | | | |
| 175 | AGAACTCTTGATTTTGTACATAGTCCTCTGGT | CCNB2 | ENSG00000157456, | NM_004701 | − |
| | CTATCTCATGAAACCTCTTCTCAGACCA | AC092757 | ENSG00000259732 | | |
| 176 | AATTGGTAAACATCATGTTCCTGATGATAACC | STK3 | ENSG00000104375 | NM_006281 | − |
| | CAGTAGCAAAAACATTTGTACTGAGTGG | | | | |
| 177 | CATCAGTCTTGGGAAATTTGAACTTTGATCAA | ZNF367 | ENSG00000165244 | NM_153695 | − |
| | CTTAACTAAAGAAGGAAGGGTAGTAAGA | | | | |
| 178 | TTAGGGCCCTACGTAATAGGCTAATTGTACT | BUB1 | ENSG00000169679 | NM_004336 | − |
| | GCTCTTAGAATGTAAGCGTTCACGAAAAT | | | | |
| 179 | GAGTCTTTGGGATACCATTAAAAAGAAGAAA | ASPM | ENSG00000066279 | NM_018136 | − |
| | ATTTCAGCCTCTACAAGTCACAACAGAAG | | | | |
| 180 | AGAGTGTGAAAAATAGGAACACGTGCTCTAC | AURKA | ENSG00000087586 | NM_003600 | − |
| | CTCCATTTAGGGATTTGCTTGGGATACAG | | | | |
| 181 | AACTTTTTAGGGCAAAGTTAACACTGAAAGT | UTP23 | ENSG00000147679, | NM_006265 | − |
| | TCTAGCTTAAGTGTTGAAACTTTTGTGGG | RAD21 | ENSG00000164754 | | |
| 182 | ACTTAGCATTTTCTGCATCTCCACTTGGCATT | PGK1 | ENSG00000102144, | NM_000291 | − |
| | AGCTAAAACCTTCCATGTCAAGATTCAG | OPHN1 | ENSG0000079482, | | |
| | | AC010422 | ENSG00000269693 | | |
| 183 | TTTTGATGAGAATGAATCTTGGTACTTAGATG | CP | ENSG00000047457, | NM_000096 | − |
| | ACAACATCAAAACATACTCTGATCACCC | LRRC69 | ENSG00000214954, | | |
| | | AC104966 | ENSG00000253525 | | |
| 184 | TTCCCTTCAATACTCCTAAAACCAAAGAAGG | AC079781 | ENSG00000284707, | NM_183356 | − |
| | ATATTACTACCGTCAAGTCTTTGAACGCC | ASNS | ENSG00000070669 | | |
| 185 | TCCTGTCCTGCTCATTATGCCACTTCCTTTTA | CA9 | ENSG00000107159 | NM_001216 | − |
| | ACTGCCAAGAAATTTTTTAAAATAAATA | | | | |
| 186 | CAAAAACTCAGATCTATCTTAAGAGTGACCA | AL451164 | ENSG00000278419, | NM_014889 | − |
| | GGAAGAGGTTCATTGAAATAATCATGCAT | PITRM1 | ENSG00000107959 | | |
| 187 | CATACGGTTTTGTTTGGAGGATGGCTTCTGC | TMEM74B | ENSG00000125895 | NM_018354 | − |
| | TGCTAAAAATACAAAAGTTTGGAAACCGC | | | | |
| 188 | CAGAGGGACCTTATTTAAACATAAGTGCTGT | ESM1 | ENSG00000164283 | NM_007036 | − |
| | GACTTCGGTGAATTTTCAATTTAAGGTAT | | | | |
| 189 | GTTTGTGAAACTGTTAAGGTCCTTTCTAAATT | CCNE2 | ENSG00000175305 | NM_057749 | − |
| | CCTCCATTGTGAGATAAGGACAGTGTCA | | | | |
| 190 | TTAACCAGCTGTAAAACACAGACCTTTATCAA | EGLN1 | ENSG00000135766 | NM_022051 | − |
| | GAGTAGGCAAAGATTTTCAGGATTCATA | | | | |
| 191 | GGGGATGAATAGAAAACCTGTAAGCTTTGAT | CENPA | ENSG00000115163 | NM_001809 | − |
| | GTTCTGGTTACTTCTAGTAAATTCCTGTC | | | | |
| 192 | GTGATAAAGTACCTGATCCAAATGTTATGAG | LIN9 | ENSG00000183814 | NT_004559 | − |
| | AATACTGGACGAGAATTGAACGAAATTGA | | | | |
| 193 | TGCAGCAGTACTACTGTCAACATAGTGTAAA | PRC1 | ENSG00000198901 | NM_199413 | − |
| | TGGTTCTCAAAAGCTTACCAGTGTGGACT | | | | |
| 194 | GCATGAGTCACAATTACAAAGTTTTGAGCGG | PALM2-AKAP2 | ENSG00000157654; | NM_147150 | − |
| | TTTTGTAATTTGACATTTAGGAAAGTCTC | AKAP2 | ENSG00000241978 | | |
| 195 | TTATTCGAAGACACAGAAGTTGGGCAAGTCA | NMU | ENSG00000109255 | NM_006681 | − |
| | AATGTTGTGTCGTCAGTTGTGCATCCGTT | | | | |
| 196 | TGTACTGGCAGGCTCGTTTTACCTGATTCTA | PITRM1 | ENSG00000107959 | NM_014889 | − |
| | GAATATTTAAGAATCTAAAAATAAAGGGC | | | | |
| 197 | GTGGCCTATAACTTACTTGTCAACAACTGTG | HRASLS | ENSG00000127252 | NM_020386 | − |
| | AACATTTTGTGACATTGCTTCGCTATGGA | | | | |
| 198 | CCAGGACGCCACTCATTTCATCTCATTTAAG | IGFBP5 | ENSG00000115461 | NM_000599 | − |
| | GGAAAAATATATATCTATCTATTTGAGGA | | | | |
| 199 | CGGAGCGCAGGGTACTTGGCGTATAATAAGC | JHDM1D-AS1 | ENSG00000260231 | NT_007914 | − |
| | CATCAATAATTTATGGGTGAAATTGAGAG | | | | |
| 200 | CAGAGCTACAACTAGGAAAATTAGAGTGGTA | MSANTD3 | ENSG00000066697 | NM_080655 | − |
| | GTAGTCACTTATTTAAGAATTCATTCAGG | | | | |
| 201 | TTGGTAGTTAACCCTAACTACTTGCTCGAAG | MCM6 | ENSG00000076003 | NM_005915 | − |
| | ATTGAGATAGTGAAAGTAACTGACCAGAG | | | | |
| 202 | GCGTGAGCATGTCAGTATTCTAGTCCAGTAT | SMIM5 | ENSG00000204323 | XM_946181 | − |
| | TTGCCAGTTTCCAAGTAAAAGCTTTTGTG | | | | |
| 203 | GCTGTGCCATTCAATGTTTGATGCATAATTG | CDCA7 | ENSG00000144354 | NM_031942 | − |
| | GACCTTGAATCGATAAGTGTAAATACAGC | | | | |
| 204 | CCAAGAAGGAAAATGTCAAAATTAGTGATGA | RFC4 | ENSG00000163918 | NM_181573 | − |
| | GGGAATAGCTTATCTTGTTAAAGTGTCAG | | | | |
| 205 | TGCTTTAAGTGAATGGCAGTCCCTTGTCTTAT | ORC6 | ENSG00000091651 | NM_014321 | − |
| | TCAGAATATAAAATTCAGTCTGAATGGC | | | | |
| 206 | AGGTTGGCAGTAAGGCAGGGTCCCATTTCTC | SLC2A3 | ENSG00000059804 | NM_006931 | − |
| | ACTGAGAAGATTGTGAATATTTCCATATG | | | | |
| 207 | GTGCAAATAGAATTAGCAGTAAGAAGCTACT | ADGRG6 | ENSG00000112414 | NM_1032395 | − |
| | CTAGCTAATTTGCCATTTCACTTAAATGG | | | | |
| 208 | GATACAGCCTACATAAAGACTGTTATGATCG | MELK | ENSG00000165304 | NM_014791 | − |
| | CTTTGATTTTAAAGTTCATTGGAACTACC | | | | |
| 209 | CAACATTTACATTGTAATTCAATAGACGCTAC | GRHL2 | ENSG00000083307 | NT_008046 | − |
| | TACTACAAAGGAGCTTTATTCTTCCAGC | | | | |
| 210 | CAACAGTATTGCGTTGTCAGACTAGGAAAGC | MTDH | ENSG00000147649 | NM_178812 | − |
| | TAAACGAACAAAATGGTTTTAGTTTTGCT | | | | |
| 211 | CTGGTTGTCCAACTACCATATGAAGCTAGAA | UCHL5 | ENSG00000116750 | NM_015984 | − |
| | AATGCACAAACGATATTCCTTATCTGTAA | | | | |
| 212 | GGCATCAGGGATCACATCACTCTTAACGGCT | RAB6B | ENSG00000154917 | NM_016577 | − |
| | GTTACTTAAACAACTATTTTTTGGTTTGG | | | | |
| 213 | TGAAAATGTATTTGTAGTCACGGACTTTCAG | ECT2 | ENSG00000114346 | NM_018098 | − |
| | GATTCTGTCTTTAATGACCTCTACAAGGC | | | | |
| 214 | AGACCAGGTCTCTATTTTGAGGAAGAAATAC | EXT1 | ENSG00000182197 | NM_000127 | − |
| | CGAGACATTGAGCGACTTTGAGGAATCCG | | | | |
| 215 | AAGTCATGACACAGTATTCGCTCTTTTTCTGA | GPR180 | ENSG00000152749 | NM_180989 | − |
| | ATGTTTACATAGAGATTCATCACTGCAG | | | | |
| 216 | CAGTAAGTACGGGAAAAAATGTTTACTAACT | LPCAT1 | ENSG00000153395 | NM_024830 | − |
| | TCCTCAGAGATTCGTGATACGCGTTTCTC | | | | |
| 217 | CTTTGAATGGACATAAAAATTCTGCTTGTTAA | SERFIA | ENSG00000172058 | NM_021967 | − |
| | GAACAAGTTGAGCTCTGGTAACTGATCT | | | | |
| 218 | TGACTGATGTGTCTGAAAATGCTAAGGATCT | CDC42BPA | ENSG00000143776 | NM_003607 | − |
| | TATTCGAAGGCTCATTTGTAGCAGAGAAC | | | | |
| 219 | CTCTGAAAGAAGAAGTTCAAAAGCTGGATGA | NDC80 | ENSG00000080986 | NM_006101 | − |
| | TCTTTACCAACAAAAAATTAAGGAAGCAG | | | | |
| 220 | ATCTGTGGTTATTCGAACCTTTATTACTAGTG | GMPS | ENSG00000163655 | NM_003875 | − |
| | ACTTCATGACTGGTATACCTGCAACACC | | | | |
| 221 | TCCACCCCAGGACGCCACTCATTTCATCTCAT | IGFBP5 | ENSG00000115461 | NM_000599 | − |
| | TTAAGGGAAAAATATATATCTATCTATT | | | | |
| 222 | GGCCCTCTCTTCTCACCTTTGTTTTTTGTTGG | MMP9 | ENSG00000100985 | NM_004994 | − |
| | AGTGTTTCTAATAAACTTGGATTCTCTA | | | | |
| 223 | CTGGGTTGATACCTGAAAGAATCCTGTCTTA | CMC2 | ENSG00000103121 | NM_020188 | − |
| | TTTGGTCTCCATAATCCTTTGAATGGAAA | | | | |
| 224 | AGTACCCTGATATACTGAATTTTGTGGATGAT | DIAPH3 | ENSG00000139734 | NM_030932 | − |
| | TTGGAACCTTTAGACAAAGCTAGTAAAG | | | | |
| 225 | AAGACTTTCTTACTGACCTGAATAACTTCAGA | DIAPH3 | ENSG00000139734 | NM_030932 | − |
| | ACCACATTCATGCAAGCAATAAAGGAGA | | | | |
| 226 | TTTAGTGGTCCGTTGCCTCTGAAGATGTAAA | QSOX2 | ENSG00000165661 | NM_181701 | − |
| | CAAACAAATACACTATTTCTGGGAACATT | | | | |
| 227 | ATAGAATATGTATATGTATTCTTTGTCTACCA | TMEM65 | ENSG00000164983 | NT_008046 | − |
| | ACTACCAAAGAAACAAATACTCCTCAGT | | | | |
| 228 | ACATTGCTTACTTAAAAGCTACATAGCCCTAT | NUSAP1 | ENSG00000137804 | NM_018454 | − |
| | CGAAATGCGAGGATTAATGCTTTAATGC | | | | |
| 229 | ACCATAAGGCAATTGAGCACATAACGAAAAA | DIAPH3 | ENSG00000139734 | NM_001042517 | − |
| | TGATGCAATAAGAATGTATGCACTCTCTT | | | | |
| 230 | CAGCCTTTCCTCATGTCAACACAGTTCACAAT | MIR210HG | ENSG00000247095 | NT_035113 | − |
| | ATAGTTTTCAAAGTACAGTTTAAAACTC | | | | |
| 231 | CCTCCCCAAAATAATTAGTAACTGGTTGTTCT | TSPYL5 | ENSG00000180543 | NM_033512 | − |
| | ACTTGGTAATTTGACACCCTGTTAATAA | | | | |

TABLE 2
Preferred genes.

| | Gene | | | Gene | |
|---|---|---|---|---|---|
| | Name | Description | | Name | Description |
| 194 | PALM2- | ENSG00000157654; | 161 | DTL | ENSG00000143476 |
| | AKAP2; AKAP2 | ENSG00000241978 | | | |
| 1 | ALDH4A1 | ENSG00000159423 | 5 | EBF4 | ENSG00000088881 |
| 17 | AP2B1 | ENSG00000006125 | 12 | ECI2 | ENSG00000198721 |
| 4 | BBC3 | ENSG00000105327 | 15 | ECI2 | ENSG00000198721 |
| 174 | C16orf95 | ENSG00000260456 | 216 | ECT2 | ENSG00000114346 |
| 3 | CAPZB | ENSG00000077549 | 184 | EGLN1 | ENSG00000135766 |
| 189 | CCNE2 | ENSG00000175305 | 182 | ESM1 | ENSG00000164283 |
| 218 | CDC42BPA | ENSG00000143776 | 222 | EXT1 | ENSG00000182197 |
| 203 | CDCA7 | ENSG00000144354 | 2 | FGF18 | ENSG00000156427 |
| 191 | CENPA | ENSG00000115163 | 221 | FLT1 | ENSG00000102755 |
| 223 | CMC2 | ENSG00000103121 | 215 | GMPS | ENSG00000163655 |
| 162 | COL4A2 | ENSG00000134871 | 220 | GNAZ | ENSG00000128266 |
| 160 | DCK | ENSG00000156136 | 201 | ADGRG6 | ENSG00000112414 |
| 18 | DHX58 | ENSG00000108771 | 210 | GPR180 | ENSG00000152749 |
| 229 | DIAPH3 | ENSG00000139734 | 206 | GRHL2 | ENSG00000083307 |
| 224 | DIAPH3 | ENSG00000139734 | 9 | GSTM3 | ENSG00000134202 |
| 225 | DIAPH3 | ENSG00000139734 | 212 | SERF1A | ENSG00000172058 |
| 191 | HRASLS | ENSG00000127252 | 196 | PITRM1 | ENSG00000107959 |
| 198 | IGFBP5 | ENSG00000115461 | 193 | PRC1 | ENSG00000198901 |
| 221 | IGFBP5 | ENSG00000115461 | 226 | QSOX2 | ENSG00000165661 |
| 199 | JHDM1D- | ENSG00000260231 | 212 | RAB6B | ENSG00000154917 |
| | AS1 | | | | |
| 192 | LIN9 | ENSG00000183814 | 204 | RFC4 | ENSG00000163918 |
| 216 | LPCAT1 | ENSG00000153395 | 11 | RTN4RL1 | ENSG00000185924 |
| 201 | MCM6 | ENSG00000076003 | 42 | RUNDC1 | ENSG00000198863 |
| 208 | MELK | ENSG00000165304 | 65 | SCUBE2 | ENSG00000175356 |
| 230 | MIR210HG | ENSG00000247095 | 206 | SLC2A3 | ENSG00000059804 |
| 222 | MMP9 | ENSG00000100985 | 202 | SMIM5 | ENSG00000204323 |
| 16 | MS4A7; | ENSG00000166927; | 14 | STK32B | ENSG00000152953 |
| | MS4A14 | ENSG00000110079 | | | |
| 200 | MSANTD3 | ENSG00000066697 | 13 | TGFB3 | ENSG00000119699 |
| 210 | MTDH | ENSG00000147649 | 227 | TMEM65 | ENSG00000164983 |
| 219 | NDC80 | ENSG00000080986 | 187 | TMEM74B | ENSG00000125895 |
| 195 | NMU | ENSG00000109255 | 231 | TSPYL5 | ENSG00000180543 |
| 228 | NUSAP1 | ENSG00000137804 | 211 | UCHL5 | ENSG00000116750 |
| 205 | ORC6 | ENSG00000091651 | 8 | WISP1 | ENSG00000104415 |
| 84 | OXCT1 | ENSG00000083720 | 38 | ZNF385B | ENSG00000144331 |

### 5 EXAMPLES

### Example 1

### Outline of the Study

A total of 372 breast cancer samples of patients with breast cancer having a High Risk MammaPrint outcome were entered into an I-SPY 2 trial. Additional criteria were a tumor size larger than 2.5 cm, adequate organ function as measured by a Performance Status (PS) score <2 (Eastern Cooperative Oncology Group scale; Oken et al., 1982. Am J Clin Oncol 5: 649-655), agreement to undergo MRI analyses and surgery following chemotherapy, and consent to analysis of biopsy samples.

A total of 299 MammaPrint High Risk patients were randomized as controls and treated with standard chemotherapy: First paclitaxel 80 mg/m2 every week for 12 weeks, followed by doxorubicin (60 mg/m2) and cyclophosphamide (600 mg/m2) for 4 cycles every 3 weeks.

A total of 73 MammaPrint High Risk patients were concurrently randomized to receive a combination of duvalumab and olaparib, in addition to paclitaxel 80 mg/m2 every week for 12 weeks, followed by doxorubicin (60 mg/m2) and cyclophosphamide (600 mg/m2) for 4 cycles every 3 weeks. Specifically, duvalumab 1500 mg was administered every 4 weeks for a total of 3 cycles; olaparib 100 mg was administered twice daily in the first 12 weeks.

The MammaPrint high risk group was further divided into 2 groups by taking the median MammaPrint index of the high risk samples (MP1 and MP2). MammaPrint high risk group 2 (MP2) is defined as having a MammaPrint index higher than the median value, while MP1 is defined as having a MammaPrint index lower than the median value (Wolf et al., 2017. NPJ Breast Cancer 3: 1-9). In total 209 patients were analyzed, 132 HR+HER2− patients were classified as MP1 of which 24 were randomized to the Duvalumab/Olaparib arm and 108 to the control arm. The remaining 77 HR+HER2− patients were classified as MP2. 28 of the MP2 patients were randomized to the Duvalumab/Olaparib arm and 49 to the control arm.

Patient and baseline clinical characteristics, ethnicity, HR status, tumor size and nodal status (see Table 3), were similar between the experimental and control arms.

The primary endpoint was pathologic complete response @CR), i.e., no invasive cancer left in the breast or lymph nodes. Hormone receptor (HR; progesterone and estradiol receptor) status and HER2 status were determined by molecular subtyping (e.g. BluePrint; Krijgsman et al., 2012. Breast Cancer Res Treat 133: 37-47; Mittempergher et al., 2020. Transl Oncol. 13: 100756); immunohistochemistry and/or fluorescent in situ hybridization.

Patients who received non-protocol therapy, left their treating institution, or withdrew consent prior to surgery were considered non-pCR as per protocol.

### Results

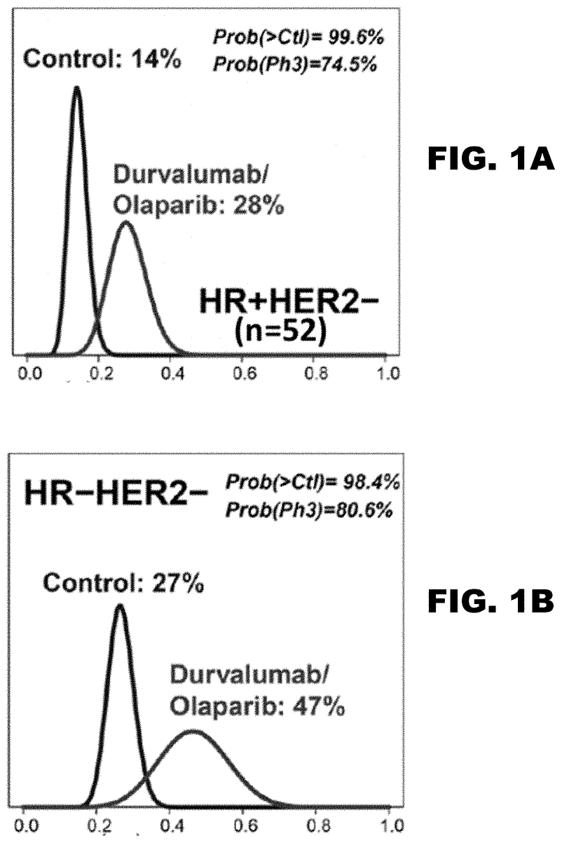

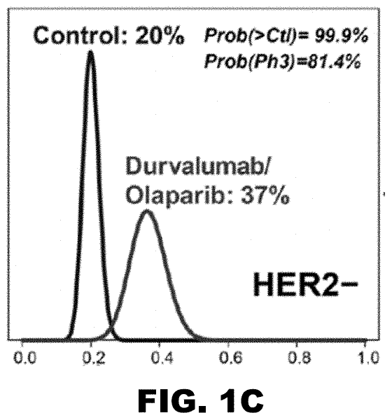

MammaPrint high risk classification associated with response in the Duvalumab/Olaparib arm in in Her2 negative subtype, but not in the control. Bayesian modeling (Gelman et al., 2019. J American Statistician 73: 307-309) was used to estimate the pCR probabilities to Duvalumab/Olaparib and standard chemotherapy in the HER2 negative subset. This was performed similar to the I-SPY 2 primary efficacy analysis (Barker et al., 2009. Clin Pharmacol Therapeutics 86: 97-100). As shown in FIG. 1 and Table 4, estimated pCR probability in the Duvalumab/Olaparib arm is 37% vs. 20% in the control arm.

TABLE 3
Patient characteristics.

| | | |
|---|---|---|
| | Durvalumab/ | |
| | Olaparib | Control |
| Patient characteristics | (n = 73) | (n = 299) |

| | | | | |
|---|---|---|---|---|
| Median Age, yrs (range) | 46 | (28-71) | 48 | (24-80) |

Race, n (%)

| | | | | |
|---|---|---|---|---|
| White | 59 | (81%) | 234 | (78%) |
| African America | 8 | (11%) | 40 | (13%) |
| Asian | 6 | (8%) | 22 | (7%) |
| Other | 0 | (0%) | 3 | (1%) |

HR status, n (%)

| | | | | |
|---|---|---|---|---|
| Positive | 52 | (71%) | 157 | (53%) |
| Negative | 21 | (29%) | 142 | (47%) |
| Median tumor size by MR, cm | 3.7 | (1.9-13) | 3.8 | (1.2-15) |
| (range) | | | | |

Palpable nodes, n (%)

| | | | | |
|---|---|---|---|---|
| Yes | 21 | (29%) | 109 | (36%) |
| No | 19 | (26%) | 129 | (43%) |
| Missing | 33 | (45%) | 61 | (20%) |

The Her2 negative subtype was further analyzed for hormone positive and hormone negative subtypes. MammaPrint High Risk classification in the Hormone negative and in the HER2 negative subgroup associated with response in the Duvalumab/Olaparib arm, but not in the control. Similarly, in the Hormone positive, HER2 negative, subtype, MammaPrint High Risk classification associated with response in the Duvalumab/Olaparib arm, but not in the control group.

Bayesian modeling was also used to estimate the pCR probabilities to Duvalumab/Olaparib and standard chemotherapy in the Hormone negative, HER2 negative subtype (HR−Her2−). As is shown in FIG. 1 and Table 4, this probability is 47% vs. 27% and the probability is 28% in the Hormone positive, HER2 negative subtype (HR+/HER2−) versus 14% in the control group.

Table 4 shows the probability of Duvalumab/Olaparib demonstrating superiority to control in a 1:1 randomized neoadjuvant phase 3 trial of 300 biomarker predicted-sensitive patients. These probabilities are >98% (99.9% for MammaPrint High Risk in Her2 negative subgroup, 98.4% for MammaPrint high risk in the HR−/Her2− subtype, 99.6% for MammaPrint High risk in the HR+/Her2-subtype).

The predictive probability for success in a randomized phase 3 trial is 81.4% in the HER2 negative subtype, 80.6 in the Her2negative/hormone negative subtype and 74.5% in the Hormone positive/HER2negative subtype.

TABLE 4
pCR scores and probability of Duvalumab/Olaparib versus control.

| | | | | Predicted |
|---|---|---|---|---|
| | | | | probability |
| | Estimated pCR rate | | | of success |
| | (95% probability interval | | Probability | in 300 patient |
| | Duvalumab/ | | superior to | randomized |
| Signature | Olaparib | Control | control | trial |
| HER2− | 0.367 | 0.201 | 0.999 | 0.814 |
| | (0.27-0.47) | (0.16-0.25) | | |
| TNBC | 0.466 | 0.267 | 0.984 | 0.806 |
| | (0.29-0.64) | (0.20-0.34) | | |
| HR+/HER2− | 0.282 | 0.143 | 0.996 | 0.745 |
| | (0.18-0.38) | (0.09-0.19) | | |

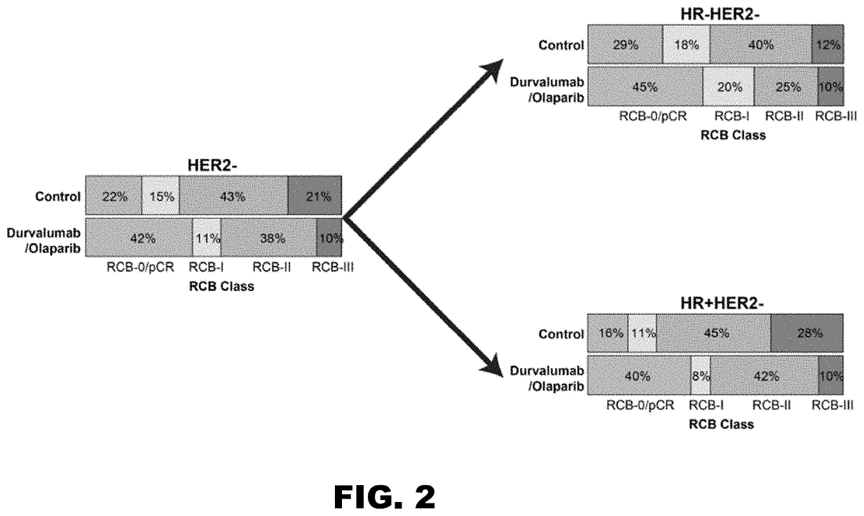

Residual Cancer Burden (RCB) after neoadjuvant chemotherapy has been shown to be an accurate long-term predictor of disease recurrence and survival across all breast cancer subtypes (Symmans et al., 2007. J Clin Oncol 24: 536). In addition to complete response, RCB is reduced in patients that were MammaPrint high risk and treated with Duvalumab/Olaparib. RCB shifted to lower values across all RCB categories in all subtypes, except RCBIII in HR−/HER2− subtype (FIG. 2).

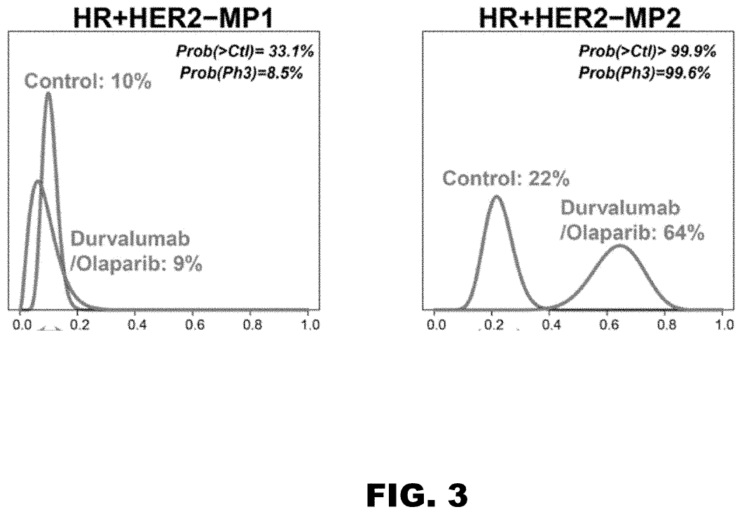

MammaPrint high risk 2 group (MP2) drives the benefit of Duvalumab/Olaparib in the HR+HER2− subtype (FIG. 3). In HR+HER2− subtypes, MP2 associated with response in the Duvalumab/Olaparib, but not in the control groups, nor in the MP1 group. Bayesian modeling was performed to estimate the pCR probabilities to Duvalumab/Olaparib treatment and to standard chemotherapy. As shown in FIG. 1, estimated pCR probability for patients classified as MammaPrint High risk 2 group (MP2) in the Duvalumab/Olaparib arm is 64% vs. 22% in the control arm. Patients classified as MP1 did not show any benefit from Duvalumab/Olaparib treatment in addition to standard chemotherapy (p CR probability 9% vs 10% in the control group).

The probability of MP2 patients treated with Duvalumab/Olaparib demonstrating superiority to control in a 1:1 randomized neoadjuvant phase 3 trial of 300 biomarker predicted-sensitive patients was calculated. These probabilities are 99.9% for MammaPrint High Risk2 (MP2) in HR+HER2− subgroup, and 33.1% in the MP1 subgroup (FIG. 3). The predictive probability for success in a randomized phase 3 trial is 99.6% in the MP2 group, and only 8.5% in the MP1 group. These numbers indicate that MP2 is driving the benefit of Duvalumab/Olaparib.

1-15. (canceled)

16. A method of treating an individual, comprising administering to an individual with human epidermal growth factor receptor 2 (HER2) negative, MammaPrint high risk 2 (MP2) breast cancer, a poly [ADP-ribose] polymerase (PARP) inhibitor, an immune check point inhibitor, or a combination of a PARP inhibitor and an immune check point inhibitor.

17. The method of claim 16, wherein the PARP inhibitor is selected from the group consisting of olaparib, rucaparib, pamiparib, niraparib and talazoparib.

18. The method of claim 16, wherein the immune check point inhibitor is selected from the group consisting of tremelimumab, pembrolizumab, nivolumab, pidilizumab, cemiplimab, atezolizumab, avelumab and durvalumab.

19. The method of claim 16, comprising administering to the individual a combination of a PARP inhibitor and an immune check point inhibitor.

20. The method of claim 19, wherein the PARP inhibitor is administrated simultaneously with, separately from, or sequentially to the immune check point inhibitor.

21. The method of claim 16, wherein the method further comprises administering a taxane.

22. The method of claim 16, wherein the method further comprises administering a taxane selected from the group consisting of paclitaxel, docetaxel, and cabazitaxel.

23. The method of claim 16, wherein the individual has a HER2 negative, ER positive breast cancer.

24. The method of claim 16, wherein the HER2 status is determined by TargetPrint or by BluePrint.

25. A pharmaceutical composition comprising a PARP inhibitor and an immune check point inhibitor.

26. The pharmaceutical composition according to claim 25, wherein the PARP inhibitor is selected from the group consisting of olaparib, rucaparib, pamiparib, niraparib and talazoparib.

27. The pharmaceutical composition according to claim 25, wherein the immune check point inhibitor is selected from from the group consisting of tremelimumab, pembrolizumab, nivolumab, pidilizumab, cemiplimab, atezolizumab, avelumab and durvalumab.

28. The pharmaceutical composition according to claim 25, further comprising a taxane.

29. The pharmaceutical composition according to claim 25, further comprising a taxane selected from the group consisting of paclitaxel, docetaxel, and cabazitaxel.


SEQUENCE LISTING (231 sequences)

SEQ ID NO: 1 - DNA, 60 bp, Artificial Sequence
  ALDH4A1 probe
ctgagtggtcagagatctgtaaagcatgactttcaaggatggttcttaggggactgtgta

SEQ ID NO: 2 - DNA, 60 bp, Artificial Sequence
  FGF18 probe
aggacttgaatgaggaaaccaacactttgagaaaccaaagtcctttttcccaaaggttct

SEQ ID NO: 3 - DNA, 60 bp, Artificial Sequence
  CAPZB probe
gccattaagatttggatgggaagttatgggtaatgagaatataatgacatcttgcaacat

SEQ ID NO: 4 - DNA, 60 bp, Artificial Sequence
  BBC3 probe
gatggcccagcctgtaagatactgtatatgcgctgctgtagataccggaatgaattttct

SEQ ID NO: 5 - DNA, 60 bp, Artificial Sequence
  EBF4 probe
ggcctcacattctgctctgctaagtttggagaaaacagaacaataaaccagatgcaggtg

SEQ ID NO: 6 - DNA, 60 bp, Artificial Sequence
  NT_022517 probe
aagtactggaatgtaatggttgaaattcctattcagtgatctggaagaactctaatgttc

SEQ ID NO: 7 - DNA, 60 bp, Artificial Sequence
  MYLIP probe
ccaacgcacaccagtcttctcaatctgactgtaatctaatctgttgtgcttttgttggac

SEQ ID NO: 8 - DNA, 60 bp, Artificial Sequence
  WISP1 probe
ggtttaaagctgaagaggttgaagctaaaaggaaaaggttgttgttaatgaatatcaggc

SEQ ID NO: 9 - DNA, 60 bp, Artificial Sequence
  GSTM3 probe
ggctaaaagggaaaaaggatatgtggagaatcatcaagatatgaattgaatcgctgcgat

SEQ ID NO: 10 - DNA, 60 bp, Artificial Sequence
  RAB27B, AC098848.1 probe
cctttcaaacatgatcaaagatttcccaatgtgatctcatcatcatggatactcaatttg

SEQ ID NO: 11 - DNA, 60 bp, Artificial Sequence
  RTN4RL1 probe
ggggaacaatgagggcatttcatgaaccatctcaggcacttctgcatcacggaagacctg

SEQ ID NO: 12 - DNA, 60 bp, Artificial Sequence
  ECI2 probe
tgccttgagaatttcaaaagaggtaatcaggaaaagagagagagaaaaactacacgctgt

SEQ ID NO: 13 - DNA, 60 bp, Artificial Sequence
  TGFB3 probe
gtctgggattaagggcaaatctattacttttgcaaactgtcctctacatcaattaacatc

SEQ ID NO: 14 - DNA, 60 bp, Artificial Sequence
  STK32B probe
taaaaaagaaatagtcagtgttttcctcctttcaaccgagactatttctggattgtgtgc

SEQ ID NO: 15 - DNA, 60 bp, Artificial Sequence
  ECI2 probe
ttttcagaaagaagtctggaccaggctgaaggcatttgcaaagcttcccccaaatgtctt

SEQ ID NO: 16 - DNA, 60 bp, Artificial Sequence
  MS4A7,MS4A14 probe
cctcattgccttattcggagtactattatccaatatatgaaatcaaagattgtctcctga

SEQ ID NO: 17 - DNA, 60 bp, Artificial Sequence
  AP2B1 probe
tggcatcatacaaagagcaggagaagcaaacacccagaactcttttgctggtcagagatt

SEQ ID NO: 18 - DNA, 60 bp, Artificial Sequence
  DHX58 probe
tccagacctaccttgtacgcacatagacattttcatatgcactggatggagttagggaaa

SEQ ID NO: 19 - DNA, 60 bp, Artificial Sequence
  RAI2 probe
atctttgttaattattttggggagtagttgggaaatggaaaggtgaattggctctagagg

SEQ ID NO: 20 - DNA, 60 bp, Artificial Sequence
  HIPK2 probe
gttcatttccagccctttctagatctgatcttttagggggaaagacagcttaaaatgttc

SEQ ID NO: 21 - DNA, 60 bp, Artificial Sequence
  QDPR probe
tgaatgtcatgtttatgtcatagacgtagaaaacgcatccttgaattaaactgccttaac

SEQ ID NO: 22 - DNA, 60 bp, Artificial Sequence
  ZG16B probe
tactggagtaactgagtcgggacgctgaatctgaatccaccaataaataaagcttctgca

SEQ ID NO: 23 - DNA, 60 bp, Artificial Sequence
  NEO1 probe
cagattccccagaaactaccttttgcccaaagaacatgctcagtatttggggcatttcct

SEQ ID NO: 24 - DNA, 60 bp, Artificial Sequence
  ACADS probe
aggcaggggtggtgattcatgctgtgtgactgactgtgggtaataaacacacctgtcccc

SEQ ID NO: 25 - DNA, 60 bp, Artificial Sequence
  BTG2 probe
tggatttctaaactgctcaattttgactcaaaggtgctatttaccaaacactctccctac

SEQ ID NO: 26 - DNA, 60 bp, Artificial Sequence
  BBOF1, ALDH6A1 probe
ccaatccaacaactataggctgggttaaataaaaggtcattattgtctatattccaagtg

SEQ ID NO: 27 - DNA, 60 bp, Artificial Sequence
  LYPD6,LINC00474 probe
tctaccacattaaattctccattacatctcactattggtaatggcttaagtgtaaagagc

SEQ ID NO: 28 - DNA, 60 bp, Artificial Sequence
  CIRBP probe
tgaggaattcttgtacgcagttttctttggctttacgagccgattaaaagaccgtgtgaa

SEQ ID NO: 29 - DNA, 60 bp, Artificial Sequence
  AC07914,MATN3 probe
ctggtctttgaaagaaatgtactactaaagagcactagttgtgaatttagggtgttaaac

SEQ ID NO: 30 - DNA, 60 bp, Artificial Sequence
  INPP5J probe
atgatgggagagctctggcagatgtcccaatcctggaggtcatccattaggaattaaatt

SEQ ID NO: 31 - DNA, 60 bp, Artificial Sequence
  FGD6 probe
caacttgctctttcatatgagttggtcatagcatgtaagaaccaatcttgaaatatcgtt

SEQ ID NO: 32 - DNA, 60 bp, Artificial Sequence
  CACNA1D, CHDH probe
cctggatcagagtaagaatgtcttaagaagaggtttgtaaggtcttcataacaaagtggt

SEQ ID NO: 33 - DNA, 60 bp, Artificial Sequence
  SDSL probe
ctcctggactgcttcttttggctctccgacaactccggccaataaacactttctgaattg

SEQ ID NO: 34 - DNA, 60 bp, Artificial Sequence
  MINOS1, NBL1, MINOS1-NBL1 probe
aaccaacccataattgcattttacttgtcgtggttcgatctgattgtattgtcgaaggac

SEQ ID NO: 35 - DNA, 60 bp, Artificial Sequence
  PEX12 probe
attcctttatgagctctccatatccttcttgagaaactggttaaaaaaggaataggggta

SEQ ID NO: 36 - DNA, 60 bp, Artificial Sequence
  ERGIC1 probe
agtgggggttgtgtaaaggggaagtcatcttttgagatccagatagacatggtttgtgca

SEQ ID NO: 37 - DNA, 60 bp, Artificial Sequence
  FBXO16 probe
tcagcttaagtacttattgtggtagtgagtcctacggtatttcagtaaaaaggaattcat

SEQ ID NO: 38 - DNA, 60 bp, Artificial Sequence
  ZNF385B probe
ggcaagagttatcatagaacaacaaaatagagtggactcttttagagcatctatatctgc

SEQ ID NO: 39 - DNA, 60 bp, Artificial Sequence
  IP6K2 probe
ggagtttctgtttagggcattaaaaattcccgcaaactataaagagcaatgttttcagtc

SEQ ID NO: 40 - DNA, 60 bp, Artificial Sequence
  MARCH8 probe
ataattctctgtacaggggggtttgtgctatacactgggatgtctaattgcagcaataaa

SEQ ID NO: 41 - DNA, 60 bp, Artificial Sequence
  CMTM8, KRT18P15,KRT18P34,KRT18P13,PCDH11Y, KRT18P10 probe
aggactttaatcttggtgatgccttggacagcagcaactccatgcaaaccatccaaaaga

SEQ ID NO: 42 - DNA, 60 bp, Artificial Sequence
  RUNDC1 probe
gggcaaaatgtatcactccaaacactactgattcagcattgttttcatgtcttaaaattg

SEQ ID NO: 43 - DNA, 60 bp, Artificial Sequence
  TBC1D9 probe
ctggatgtttagcttcttactgcaaaaacataagtaaaacagtcaactttaccatttccg

SEQ ID NO: 44 - DNA, 60 bp, Artificial Sequence
  LETMD1 probe
ggtaacttgcaggaatattctattggaaaagataacaggaagtacaagtgcttcttgacc

SEQ ID NO: 45 - DNA, 60 bp, Artificial Sequence
  RILPL2 probe
tcaatggttagcagaagggagaaaagaaagcaggaaaatgtgctattgagattccagtgg

SEQ ID NO: 46 - DNA, 60 bp, Artificial Sequence
  SEC14L2, AC004832.3 probe
cctgggtttacaacgctgttaggaaaattaaccaatgaataaagcaacgttcagtgcgca

SEQ ID NO: 47 - DNA, 60 bp, Artificial Sequence
  KIAA1217 probe
tttttgtaccttgtcactataactacttcctagtcaaagaacgaaatgtaactgttaccg

SEQ ID NO: 48 - DNA, 60 bp, Artificial Sequence
  CCDC74A,MED15P9,CCDC74B probe
ttctagctgttattttgctatttggcatttacataaaagcacacgatgaagcaggtatcg

SEQ ID NO: 49 - DNA, 60 bp, Artificial Sequence
  TBX3 probe
ttgggtttatttccaggtcacagaattgctgttaacactagaaaacacacttcctgcacc

SEQ ID NO: 50 - DNA, 60 bp, Artificial Sequence
  FUT8 probe
gaacagctccttactctgaggaagttgattcttatttgatggtggtattgtgaccactga

SEQ ID NO: 51 - DNA, 60 bp, Artificial Sequence
  KIF3B probe
ctttcttatttactaagaatttgcctgtttgaataagaacaaaacgctaaggtgggtagc

SEQ ID NO: 52 - DNA, 60 bp, Artificial Sequence
  PCAT7, FBP1 probe
ctagagagcagaaataaaaagcatgactatttccaccatcaaatgctgtagaatgcttgg

SEQ ID NO: 53 - DNA, 60 bp, Artificial Sequence
  LBHD1, CSKMT probe
gttcaggggcatcacctactttgcttacttgattcaaggctctcattaaagacattttag

SEQ ID NO: 54 - DNA, 60 bp, Artificial Sequence
  KIAA1324 probe
gttggtagagggagtatgataaaatgtttaaatctcatttggttaccttgagtcctggaa

SEQ ID NO: 55 - DNA, 60 bp, Artificial Sequence
  TMEM25 probe
aattcaacagtgtggaagctttaggggaacatggagaaagaaggagaccacataccccaa

SEQ ID NO: 56 - DNA, 60 bp, Artificial Sequence
  PIN4, RPS4X probe
caagttgtgcaaagtgagaaagatctttgtgggcacaaaaggaatccctcatctggtgac

SEQ ID NO: 57 - DNA, 60 bp, Artificial Sequence
  STON2 probe
caagagaacctggagaaaactaccgtattcaagagattaatcaaaatcagtgttttagcc

SEQ ID NO: 58 - DNA, 60 bp, Artificial Sequence
  TENM3 probe
ccgaatgaccttaaaggtgatcggctttaacgaatatgtttacatatgcatagcgctgca

SEQ ID NO: 59 - DNA, 60 bp, Artificial Sequence
  RASL11B probe
agtttatgggccagaatattctgtataccagacattggtaagctctcatggtttacagga

SEQ ID NO: 60 - DNA, 60 bp, Artificial Sequence
  GSDMD probe
ccatgtggccagtctaccatggggcccaggagttggggaaacacaataaaggtggcatac

SEQ ID NO: 61 - DNA, 60 bp, Artificial Sequence
  LAMP5 probe
atgcttaaacccacggaagggggagactctttcggatttgtagggtgaaatggcaattat

SEQ ID NO: 62 - DNA, 60 bp, Artificial Sequence
  CHPT1, SYCP3 probe
ttctttcttcaaagagtcatcagaataacatggattgaagagacttccgaacacttgcta

SEQ ID NO: 63 - DNA, 60 bp, Artificial Sequence
  ZNF627 probe
tgaagtcagcgttaaccatgtgcatacaacttaaggaattttttcctcctcatgtaaatt

SEQ ID NO: 64 - DNA, 60 bp, Artificial Sequence
  COL23A1 probe
gttaaacagggattatagtacttgtctcacaaagtttctgtgagaattaaacaaggggat

SEQ ID NO: 65 - DNA, 60 bp, Artificial Sequence
  SCUBE2 probe
cagcctgtgtgatacaagtttgatcccaggaacttgagttctaagcagtgctcgtgaaaa

SEQ ID NO: 66 - DNA, 60 bp, Artificial Sequence
  AC023024.1, PCSK6 probe
aatgcacagatctgcttgatcaattcccttgaatagggaagtaacatttgccttaaattt

SEQ ID NO: 67 - DNA, 60 bp, Artificial Sequence
  RBP3 probe
tttccaataaccacctaaattttaacaaaggttccttctaagtggtagaacttggggtgg

SEQ ID NO: 68 - DNA, 60 bp, Artificial Sequence
  MYRIP, EIF1B-AS1 probe
agttatgcttcccttcatgttatatgcacattgccaagaattactgtcaagagaaatgat

SEQ ID NO: 69 - DNA, 60 bp, Artificial Sequence
  SPEF1 probe
aaggtttgaaggttacggctcagggctgccccattaaagtcagtgttgtgttctaaaaaa

SEQ ID NO: 70 - DNA, 60 bp, Artificial Sequence
  CLSTN2 probe
ggactgtatgaatttatagaaaattgaatctaatttcagaagagcgcactgtcttctcag

SEQ ID NO: 71 - DNA, 60 bp, Artificial Sequence
  EVL,DEGS2 probe
tacatttctttgggtttctagagacgcccctaagtcacctgcttcattagacggtttcca

SEQ ID NO: 72 - DNA, 60 bp, Artificial Sequence
  ELMOD3 probe
ggcctaattgagggaaggaggaaattcataccagcagttttcaaataaaagaattgttct

SEQ ID NO: 73 - DNA, 60 bp, Artificial Sequence
  BBOF1, ALDH6A1 probe
tccaattctacactcagttaaagaccattacttctcagtggaaagaagaagatgctactc

SEQ ID NO: 74 - DNA, 60 bp, Artificial Sequence
  KIAA1683 probe
gtggggacttcgtgggaggcactcatggctctctgggtctaatgaataaagtcctccaca

SEQ ID NO: 75 - DNA, 60 bp, Artificial Sequence
  SPC25 probe
ccaggatcttaaggaagaatattctaggaagaaggaaactatttctactgctaataaagc

SEQ ID NO: 76 - DNA, 60 bp, Artificial Sequence
  TFRC probe
agaaaacccttttctacagttagggttgagttacttcctatcaagccagtacgtgctaac

SEQ ID NO: 77 - DNA, 60 bp, Artificial Sequence
  PAQR3 probe
tagggaatgaatgaatgaatatggattgctgttaactagaaacacttctgtatgtcagtc

SEQ ID NO: 78 - DNA, 60 bp, Artificial Sequence
  MLLT10 probe
gtacttagctggaagaacatgttaattctgcaatatgtttcttggttaaacattgcacag

SEQ ID NO: 79 - DNA, 60 bp, Artificial Sequence
  CENPBD1 probe
actctcttaggtcatttttcaatgtgtgtaaccaaaagttaatcagaataaagcggaagc

SEQ ID NO: 80 - DNA, 60 bp, Artificial Sequence
  AL44926,GPSM2, CLCC1 probe
aatgctttgttggagtttaaaaattcagggaaaaaatcggcagaccattagttactatgg

SEQ ID NO: 81 - DNA, 60 bp, Artificial Sequence
  PIMREG probe
aagaaaccagcatgtgactttcctagataacactgctttctcataataaagactatttgc

SEQ ID NO: 82 - DNA, 60 bp, Artificial Sequence
  HACD2 probe
gttggcattgatatggtacaacctgcaaattacttgcagttctgagtttcagataaaaca

SEQ ID NO: 83 - DNA, 60 bp, Artificial Sequence
  ACE, AC113554 probe
agtgtcattttaagggacatttttatgacttttatgtgtatgtttatgtagaaatttgga

SEQ ID NO: 84 - DNA, 60 bp, Artificial Sequence
  OXCT1 probe
actcacttcttttcaggtgtagctacaattgtgtaatgtacaatattagagaaaggacag

SEQ ID NO: 85 - DNA, 60 bp, Artificial Sequence
  GNAZ probe
cctgggagcaaatgaacaatagctaagtgtcttggtatttaaagagtaaattatttgtgg

SEQ ID NO: 86 - DNA, 60 bp, Artificial Sequence
  FLT1 probe
ccaagaatatatgctacagatataagacagacatggtttggtcctatatttctagtcatg

SEQ ID NO: 87 - DNA, 60 bp, Artificial Sequence
  MAD2L1,MNAT1 probe
atgctttcctaaatcagatgttttggtcaagtagtttgactcagtataggtagggagata

SEQ ID NO: 88 - DNA, 60 bp, Artificial Sequence
  CDC25B probe
atttgtgtggacaaaaatatttacacttagggtttggagctattcaagaggaaatgtcac

SEQ ID NO: 89 - DNA, 60 bp, Artificial Sequence
  KIF21A probe
aaatatactatgtttgcgaaccttggtagctatgatgagagctattatcatctgtggtgg

SEQ ID NO: 90 - DNA, 60 bp, Artificial Sequence
  HMGB3 probe
tcaatgaaagttcaagaacctcctgtacttaaacacgattcgcaacgttctgttattttt

SEQ ID NO: 91 - DNA, 60 bp, Artificial Sequence
  PTDSS1 probe
accttgatagttcaccacgtctgatggatccctgttttaaataaaaacgattcactttaa

SEQ ID NO: 92 - DNA, 60 bp, Artificial Sequence
  MTMR2 probe
taaaatacttcaatcctggattcacagtgggaacaagtttctattaaaaggcaaatgctg

SEQ ID NO: 93 - DNA, 60 bp, Artificial Sequence
  CENPU probe
ggctgtgaacaatgttaaatagcatcagtttgtccaatagttttaaaggccataatcatc

SEQ ID NO: 94 - DNA, 60 bp, Artificial Sequence
  AL353705 probe
acgagtaccggcatgttatgttacccagagaactttccaaacaagtacctaaaactcatc

SEQ ID NO: 95 - DNA, 60 bp, Artificial Sequence
  C1orf198 probe
attttttagaaaatacacacttttcaggagaaacctgagcatgattttggattctccacc

SEQ ID NO: 96 - DNA, 60 bp, Artificial Sequence
  RRM2, AC007240 probe
cagctcagaccatttcctaatcagttgaaagggaaacaagtatttcagtctcaaaattga

SEQ ID NO: 97 - DNA, 60 bp, Artificial Sequence
  INTS7 probe
cactgcagactctcaagagatcaatcaaattgccagaaacagtttggtttttcatatgga

SEQ ID NO: 98 - DNA, 60 bp, Artificial Sequence
  MRPL13 probe
tgaaactttcttctgatgagtttctttaacgtacaggatggagtaaaacaaatggtacag

SEQ ID NO: 99 - DNA, 60 bp, Artificial Sequence
  ARMC1 probe
caattcttgagagttaatgtgatcatgatattgcaaacaactataaatggtctctaggcc

SEQ ID NO: 100 - DNA, 60 bp, Artificial Sequence
  ADM probe
gaaggaaacaccgagtctctgtataatctatttacataaaatgggtgatatgcgaacagc

SEQ ID NO: 101 - DNA, 60 bp, Artificial Sequence
  IGFBP5 probe
agcaacctgggccttgtactgtctgtgtttttaaaaccactaaagtgcaagaattacatt

SEQ ID NO: 102 - DNA, 60 bp, Artificial Sequence
  SKA3 probe
gggaatttgatgcagtaaagattaccctgttttatgattgttccttgaaagtcaaatggg

SEQ ID NO: 103 - DNA, 60 bp, Artificial Sequence
  SLC7A1 probe
taaggctaatgataccaatgagggttggtttattatcaaacctgaatagctgtggtttct

SEQ ID NO: 104 - DNA, 60 bp, Artificial Sequence
  PRAME probe
tggggagatacatcttatagagttagaaatagaatctgaatttctaaagggagattctgg

SEQ ID NO: 105 - DNA, 60 bp, Artificial Sequence
  CTSV probe
tatcttgaaactgaccaaacgcttattgtgtaagataaaccagttgaatcattgaggatc

SEQ ID NO: 106 - DNA, 60 bp, Artificial Sequence
  SMC4P1, AC07959, SMC4, TRIM59 probe
ttctctgaaggaatcatgttcagtgttcgaccacctaagaaaagttggaaaaagatcttc

SEQ ID NO: 107 - DNA, 60 bp, Artificial Sequence
  NIPA1 probe
tgtcatagacatgtattggggagcttccaattagcatacatagacacatgtgtcagtggc

SEQ ID NO: 108 - DNA, 60 bp, Artificial Sequence
  SFT2D2 probe
tgtccatgctacaagaagttatgagccttgttctaagtacagatgaaccttgtatttgtg

SEQ ID NO: 109 - DNA, 60 bp, Artificial Sequence
  SACS probe
atcccgatttcagtcagacaaatactcatttcagagattctatacttcatggaatcaaga

SEQ ID NO: 110 - DNA, 60 bp, Artificial Sequence
  CTPS1 probe
agttactttcttaatgtgacctagcaataggcatagctacgtggcactatattctggcca

SEQ ID NO: 111 - DNA, 60 bp, Artificial Sequence
  NUSAP1 probe
gaaatctctctacacagatgagtcatccaaacctgggaaaaaataaaagaactgcaatca

SEQ ID NO: 112 - DNA, 60 bp, Artificial Sequence
  PSMD7, AC009120 probe
aaattgctaagtggaatgcatgaattgcattatgttctctggtaacacgtagagttcaga

SEQ ID NO: 113 - DNA, 60 bp, Artificial Sequence
  BUD23, STX1A probe
ccaaaggtcttggtacaaccagctgcccattttgtgaaatttttatgtagaataaacatt

SEQ ID NO: 114 - DNA, 60 bp, Artificial Sequence
  KIAA1147 probe
gtttcgggtctttacctcatagtatgaaattagtaagacactgcatagattttgccctga

SEQ ID NO: 115 - DNA, 60 bp, Artificial Sequence
  NDRG1 probe
gagtacggatgggaaactattgtgcacaagtctttccagaggagtttcttaatgagatat

SEQ ID NO: 116 - DNA, 60 bp, Artificial Sequence
  PFKP, AL45116 probe
tattttatcagcactttatgcacgtattattgacattaatacctaatcggcgagtgccca

SEQ ID NO: 117 - DNA, 60 bp, Artificial Sequence
  CD163L1 probe
tgccctatggaaaacttgtccaaataacatttcttgaacaataggagaacagctaaattg

SEQ ID NO: 118 - DNA, 60 bp, Artificial Sequence
  MAPRE2 probe
ctccttgtcattgaccttagctaaaccatggcaattcataaatagaggaaacattaatga

SEQ ID NO: 119 - DNA, 60 bp, Artificial Sequence
  TMEM45A probe
ctgaacgagaacaagaatcagaagaagaaatgtgactttgatgagcttccagtttttcta

SEQ ID NO: 120 - DNA, 60 bp, Artificial Sequence
  PABPC1 probe
tatattatcagtctgtaccagtagaccagtaccctaactactgaaaagaatatggcagtt

SEQ ID NO: 121 - DNA, 60 bp, Artificial Sequence
  RHBDF2 probe
agtaacgctaactttgtacggacgatgtctcatggattaaataatattctttatggcagt

SEQ ID NO: 122 - DNA, 60 bp, Artificial Sequence
  AGO2 probe
gtggatctacctcagttaaacagttgggtgctattactaagtctgtcaaattaaattgga

SEQ ID NO: 123 - DNA, 60 bp, Artificial Sequence
  TMEM64 probe
cattctaaagggaaatcagtaaaatgtcttgataattggtatccaaatcacttgtgtgcc

SEQ ID NO: 124 - DNA, 60 bp, Artificial Sequence
  MGAT4A probe
ccaaagacaaacgattagaagatggctatttcagaataggaaaatttgagaatggtgttg

SEQ ID NO: 125 - DNA, 60 bp, Artificial Sequence
  CDK16 probe
caaacttcctgacactacttccatatttgcactaaaggagattcagctacaaaaggaggc

SEQ ID NO: 126 - DNA, 60 bp, Artificial Sequence
  AL589666, SYNCRIP probe
accttcctatgaagatcatggaatcaaatacgggacattgaactaatacttggactttga

SEQ ID NO: 127 - DNA, 60 bp, Artificial Sequence
  HIF1A probe
ggctaacacaatctaattttggtttaagagacaaatctagagtctcaaatgatctcagag

SEQ ID NO: 128 - DNA, 60 bp, Artificial Sequence
  RRAGD probe
tggacccttaaatatgactaaaatcacagcaatattgttacatacgggttatatgccaac

SEQ ID NO: 129 - DNA, 60 bp, Artificial Sequence
  HIF1A probe
taagcattgtgaaggaagattaatatagccaaataactagagtgatcagttctaccagag

SEQ ID NO: 130 - DNA, 60 bp, Artificial Sequence
  DEGS1 probe
cctggataaaagtactgtatgattttgtgatggatgatacaataagtccctactcaagaa

SEQ ID NO: 131 - DNA, 60 bp, Artificial Sequence
  LRP12 probe
gctttgttactttgttaggtacgaatcacataagggagattgtatacaagttggagcaat

SEQ ID NO: 132 - DNA, 60 bp, Artificial Sequence
  ZDHHC20 probe
taaaagatgaagaaagctattaggtatatttgtacatgactgcaaatgagtctatgcccg

SEQ ID NO: 133 - DNA, 60 bp, Artificial Sequence
  PLEKHA1 probe
gtgtgttatctttatatgtcaaactggttgaacactgtaatgagaataaactgcacagag

SEQ ID NO: 134 - DNA, 60 bp, Artificial Sequence
  FBXO5 probe
gattattgtacgaagtgtctctgtaattatcatactactaaagactgttcagatggcaag

SEQ ID NO: 135 - DNA, 60 bp, Artificial Sequence
  NEAT1 probe
catttgtattaatggaatactaagtccctctgtgatttctgaaccaagctattcctaggc

SEQ ID NO: 136 - DNA, 60 bp, Artificial Sequence
  PIR probe
atgaagagatttctcaagctattcttgatttcagaaacgcaaaaaatgggtttgaaaggg

SEQ ID NO: 137 - DNA, 60 bp, Artificial Sequence
  ASPM probe
agccaatcatgagtacgtaaagtgatttttgctctctgtgtacaacttttaaaatctgac

SEQ ID NO: 138 - DNA, 60 bp, Artificial Sequence
  GBE1 probe
atcctagaccatattttcaagtcatcttagcagctaggattctcaaatggaagtgttata

SEQ ID NO: 139 - DNA, 60 bp, Artificial Sequence
  HJURP probe
agtgatttcatgctagaaaaattggaaactaaaagtgtgtagctaggttatttcggagtg

SEQ ID NO: 140 - DNA, 60 bp, Artificial Sequence
  QSER1 probe
gctaagccaagtagtagcagtaaaacttctgatcctctagcatcaaaaactacaactaca

SEQ ID NO: 141 - DNA, 60 bp, Artificial Sequence
  BNIP3 probe
ggaaagaagttgaaagcatcttgaagaaaaactcagattggatatgggattggtcaagtc

SEQ ID NO: 142 - DNA, 60 bp, Artificial Sequence
  AC087521, C11orf96, AC087521 probe
acctggatatgtctgtgaggctcctgaaaggagacaaataaagtcaatatatttgcacaa

SEQ ID NO: 143 - DNA, 60 bp, Artificial Sequence
  LINC00888 probe
gggtatgaaagatgagtgtctgtaaaaatccttcttagaaatgtatttcctcaagactct

SEQ ID NO: 144 - DNA, 60 bp, Artificial Sequence
  GGH probe
cagatggcaagattgagtttatttcaacaatggaaggatataagtatccagtatatggtg

SEQ ID NO: 145 - DNA, 60 bp, Artificial Sequence
  TRIP13 probe
gaaactgtgtcaccctaaagaagcatataatcatagcattaaaaatgcacacattactcc

SEQ ID NO: 146 - DNA, 60 bp, Artificial Sequence
  STMN1 probe
caagcgtgtttctagagaacagttgagagagaatctcaagattctacttggtggtttgct

SEQ ID NO: 147 - DNA, 60 bp, Artificial Sequence
  CENPN, AC092718, AC092718 probe
ccgacaagaggagatcattttagatattaccgaaatgaagaaagcttgcaattagtgaac

SEQ ID NO: 148 - DNA, 60 bp, Artificial Sequence
  MYO10 probe
taatagcaaaatttaacccgttactctttaaccttgtactggaaattctaagcagtgcag

SEQ ID NO: 149 - DNA, 60 bp, Artificial Sequence
  TK1 probe
cttcctacctctggtgatggtttccacaggaacaacagcatctttcaccaagatgggtgg

SEQ ID NO: 150 - DNA, 60 bp, Artificial Sequence
  RUNX1, EZH2P1 probe
aaatcattcggtaaatccaaactgctatgcaaaagttatgatggttaacggtgatcacag

SEQ ID NO: 151 - DNA, 60 bp, Artificial Sequence
  AURKA probe
ttgggtttctagtcctccttaccatcatctccatatgagagtgtgaaaataggaacacgt

SEQ ID NO: 152 - DNA, 60 bp, Artificial Sequence
  DLGAP5 probe
gctggtggagtagcagatgatattaatactaacaaaaaagaaggaatttcagatgttgtg

SEQ ID NO: 153 - DNA, 60 bp, Artificial Sequence
  TBCE, B3GALNT2 probe
tcacccagaaccaatgcggtgtttcttaatgtttgcacaaatttccttaaaaatcaactt

SEQ ID NO: 154 - DNA, 60 bp, Artificial Sequence
  CENPF probe
caggacttctctttagtcagggcatgctttattagtgaggagaaaacaattccttagaag

SEQ ID NO: 155 - DNA, 60 bp, Artificial Sequence
  AL117350, CCSAP probe
ccctgtgctatcgtaagtttgttttgagcactgcattcactttaaaattctggaggaaca

SEQ ID NO: 156 - DNA, 60 bp, Artificial Sequence
  ATAD2 probe
caacatatttcagttggaaaatttgtatgcagtaatcagccaatgtatttatcggcatcg

SEQ ID NO: 157 - DNA, 60 bp, Artificial Sequence
  PSMD2, FMN2, AL359918 probe
cccccattctggaaggttttgttatcttcggaagaaccccaattatgatctctaagtgac

SEQ ID NO: 158 - DNA, 60 bp, Artificial Sequence
  SHMT2, NDUFA4L2 probe
tgtccccagggatcaaacagaagcagccgtgggcaaaatacaatttcatttaacaaattg

SEQ ID NO: 159 - DNA, 60 bp, Artificial Sequence
  PIMREG, PITPNM3 probe
aaacagcattatggagttaaaagatttttacaactgggtcttgattttgatgtgagctgg

SEQ ID NO: 160 - DNA, 60 bp, Artificial Sequence
  DCK probe
tccagacgcactgatctttgcaaaggagacttaatttcaaatctgtaattaccatacata

SEQ ID NO: 161 - DNA, 60 bp, Artificial Sequence
  DTL probe
catttggctgtcagaaattataccgagtctactgggtataacatgtctcacttggaaagc

SEQ ID NO: 162 - DNA, 60 bp, Artificial Sequence
  COL4A2 probe
ttaaaggcaaaactgtgctctttattttaaaaaacactgataatcacactgcggtaggtc

SEQ ID NO: 163 - DNA, 60 bp, Artificial Sequence
  AGFG1 probe
aaggtgctgtcatatatcttggaatgaatgacctaaaatcattttaaccattgctactgg

SEQ ID NO: 164 - DNA, 60 bp, Artificial Sequence
  NMB probe
ggatgtaaatcctgagctcaaatctctgttactccattactgtgatttctggctgggtca

SEQ ID NO: 165 - DNA, 60 bp, Artificial Sequence
  KIF14 probe
cctcaagagtatgtataatttgaagagatactttgtaactatgcttgggtgatattgagc

SEQ ID NO: 166 - DNA, 60 bp, Artificial Sequence
  BIRC5 probe
ttcacagaatagcacaaactacaattaaaactaagcacaaagccattctaagtcattggg

SEQ ID NO: 167 - DNA, 60 bp, Artificial Sequence
  VEGFA probe
ccagcacataggagagatgagcttcctacagcacaacaaatgtgaatgcagaccaaagaa

SEQ ID NO: 168 - DNA, 60 bp, Artificial Sequence
  ECT2 probe
gagaaacattgtatattttgcaaaaacaagatgtttgtagctgtttcagagagagtacgg

SEQ ID NO: 169 - DNA, 60 bp, Artificial Sequence
  IVNS1ABP probe
tactttttggaaaagaataaaccaagaattgattgggcacatcatttcaagaagtccctc

SEQ ID NO: 170 - DNA, 60 bp, Artificial Sequence
  MCCC1 probe
atggagttgctagtaaagcgaagctgattatcctggaaaacactatttacctattttcca

SEQ ID NO: 171 - DNA, 60 bp, Artificial Sequence
  TMEM38B, AL592437, OTUD7A, AC026951, DEPDC1, AL138789 probe
gactgctagtggataataacatcttgactacttaaaaaagggacatattgaaaatcctgg

SEQ ID NO: 172 - DNA, 60 bp, Artificial Sequence
  INAVA, SLC9C1 probe
catgttacctggactggaacagactgtgaatatagcagaaggttccaagaactctggtgt

SEQ ID NO: 173 - DNA, 60 bp, Artificial Sequence
  KIF21A probe
gagaccaggtgcttcaaaacttaggctcggtagaatcttactcagaagaaaaagcaaaaa

SEQ ID NO: 174 - DNA, 60 bp, Artificial Sequence
  C16orf95 probe
ggattcaacccaaatgatttctcatcaggtgattcttggttgtagcaaagttcatgtgaa

SEQ ID NO: 175 - DNA, 60 bp, Artificial Sequence
  CCNB2, AC092757 probe
agaactcttgattttgtacatagtcctctggtctatctcatgaaacctcttctcagacca

SEQ ID NO: 176 - DNA, 60 bp, Artificial Sequence
  STK3 probe
aattggtaaacatcatgttcctgatgataacccagtagcaaaaacatttgtactgagtgg

SEQ ID NO: 177 - DNA, 60 bp, Artificial Sequence
  ZNF367 probe
catcagtcttgggaaatttgaactttgatcaacttaactaaagaaggaagggtagtaaga

SEQ ID NO: 178 - DNA, 60 bp, Artificial Sequence
  BUB1 probe
ttagggccctacgtaataggctaattgtactgctcttagaatgtaagcgttcacgaaaat

SEQ ID NO: 179 - DNA, 60 bp, Artificial Sequence
  ASPM probe
gagtctttgggataccattaaaaagaagaaaatttcagcctctacaagtcacaacagaag

SEQ ID NO: 180 - DNA, 60 bp, Artificial Sequence
  AURKA probe
agagtgtgaaaaataggaacacgtgctctacctccatttagggatttgcttgggatacag

SEQ ID NO: 181 - DNA, 60 bp, Artificial Sequence
  UTP23, RAD21 probe
aactttttagggcaaagttaacactgaaagttctagcttaagtgttgaaacttttgtggg

SEQ ID NO: 182 - DNA, 60 bp, Artificial Sequence
  PGK1, OPHN1, AC010422 probe
acttagcattttctgcatctccacttggcattagctaaaaccttccatgtcaagattcag

SEQ ID NO: 183 - DNA, 60 bp, Artificial Sequence
  CP, LRRC69, AC104966 probe
ttttgatgagaatgaatcttggtacttagatgacaacatcaaaacatactctgatcaccc

SEQ ID NO: 184 - DNA, 60 bp, Artificial Sequence
  AC079781, ASNS probe
ttcccttcaatactcctaaaaccaaagaaggatattactaccgtcaagtctttgaacgcc

SEQ ID NO: 185 - DNA, 60 bp, Artificial Sequence
  CA9 probe
tcctgtcctgctcattatgccacttccttttaactgccaagaaattttttaaaataaata

SEQ ID NO: 186 - DNA, 60 bp, Artificial Sequence
  AL451164, PITRM1 probe
caaaaactcagatctatcttaagagtgaccaggaagaggttcattgaaataatcatgcat

SEQ ID NO: 187 - DNA, 60 bp, Artificial Sequence
  TMEM74B probe
catacggttttgtttggaggatggcttctgctgctaaaaatacaaaagtttggaaaccgc

SEQ ID NO: 188 - DNA, 60 bp, Artificial Sequence
  ESM1 probe
cagagggaccttatttaaacataagtgctgtgacttcggtgaattttcaatttaaggtat

SEQ ID NO: 189 - DNA, 60 bp, Artificial Sequence
  CCNE2 probe
gtttgtgaaactgttaaggtcctttctaaattcctccattgtgagataaggacagtgtca

SEQ ID NO: 190 - DNA, 60 bp, Artificial Sequence
  EGLN1 probe
ttaaccagctgtaaaacacagacctttatcaagagtaggcaaagattttcaggattcata

SEQ ID NO: 191 - DNA, 60 bp, Artificial Sequence
  CENPA probe
ggggatgaatagaaaacctgtaagctttgatgttctggttacttctagtaaattcctgtc

SEQ ID NO: 192 - DNA, 60 bp, Artificial Sequence
  LIN9 probe
gtgataaagtacctgatccaaatgttatgagaatactggacgagaattgaacgaaattga

SEQ ID NO: 193 - DNA, 60 bp, Artificial Sequence
  PRC1 probe
tgcagcagtactactgtcaacatagtgtaaatggttctcaaaagcttaccagtgtggact

SEQ ID NO: 194 - DNA, 60 bp, Artificial Sequence
  PALM2-AKAP2, AKAP2 probe
gcatgagtcacaattacaaagttttgagcggttttgtaatttgacatttaggaaagtctc

SEQ ID NO: 195 - DNA, 60 bp, Artificial Sequence
  NMU probe
ttattcgaagacacagaagttgggcaagtcaaatgttgtgtcgtcagttgtgcatccgtt

SEQ ID NO: 196 - DNA, 60 bp, Artificial Sequence
  PITRM1 probe
tgtactggcaggctcgttttacctgattctagaatatttaagaatctaaaaataaagggc

SEQ ID NO: 197 - DNA, 60 bp, Artificial Sequence
  HRASLS probe
gtggcctataacttacttgtcaacaactgtgaacattttgtgacattgcttcgctatgga

SEQ ID NO: 198 - DNA, 60 bp, Artificial Sequence
  IGFBP5 probe
ccaggacgccactcatttcatctcatttaagggaaaaatatatatctatctatttgagga

SEQ ID NO: 199 - DNA, 60 bp, Artificial Sequence
  JHDM1D-AS1 probe
cggagcgcagggtacttggcgtataataagccatcaataatttatgggtgaaattgagag

SEQ ID NO: 200 - DNA, 60 bp, Artificial Sequence
  MSANTD3 probe
cagagctacaactaggaaaattagagtggtagtagtcacttatttaagaattcattcagg

SEQ ID NO: 201 - DNA, 60 bp, Artificial Sequence
  MCM6 probe
ttggtagttaaccctaactacttgctcgaagattgagatagtgaaagtaactgaccagag

SEQ ID NO: 202 - DNA, 60 bp, Artificial Sequence
  SMIM5 probe
gcgtgagcatgtcagtattctagtccagtatttgccagtttccaagtaaaagcttttgtg

SEQ ID NO: 203 - DNA, 60 bp, Artificial Sequence
  CDCA7 probe
gctgtgccattcaatgtttgatgcataattggaccttgaatcgataagtgtaaatacagc

SEQ ID NO: 204 - DNA, 60 bp, Artificial Sequence
  RFC4 probe
ccaagaaggaaaatgtcaaaattagtgatgagggaatagcttatcttgttaaagtgtcag

SEQ ID NO: 205 - DNA, 60 bp, Artificial Sequence
  ORC6 probe
tgctttaagtgaatggcagtcccttgtcttattcagaatataaaattcagtctgaatggc

SEQ ID NO: 206 - DNA, 60 bp, Artificial Sequence
  SLC2A3 probe
aggttggcagtaaggcagggtcccatttctcactgagaagattgtgaatatttccatatg

SEQ ID NO: 207 - DNA, 60 bp, Artificial Sequence
  ADGRG6 probe
gtgcaaatagaattagcagtaagaagctactctagctaatttgccatttcacttaaatgg

SEQ ID NO: 208 - DNA, 60 bp, Artificial Sequence
  MELK probe
gatacagcctacataaagactgttatgatcgctttgattttaaagttcattggaactacc

SEQ ID NO: 209 - DNA, 60 bp, Artificial Sequence
  GRHL2 probe
caacatttacattgtaattcaatagacgctactactacaaaggagctttattcttccagc

SEQ ID NO: 210 - DNA, 60 bp, Artificial Sequence
  MTDH probe
caacagtattgcgttgtcagactaggaaagctaaacgaacaaaatggttttagttttgct

SEQ ID NO: 211 - DNA, 60 bp, Artificial Sequence
  UCHL5 probe
ctggttgtccaactaccatatgaagctagaaaatgcacaaacgatattccttatctgtaa

SEQ ID NO: 212 - DNA, 60 bp, Artificial Sequence
  RAB6B probe
ggcatcagggatcacatcactcttaacggctgttacttaaacaactattttttggtttgg

SEQ ID NO: 213 - DNA, 60 bp, Artificial Sequence
  ECT2 probe
tgaaaatgtatttgtagtcacggactttcaggattctgtctttaatgacctctacaaggc

SEQ ID NO: 214 - DNA, 60 bp, Artificial Sequence
  EXT1 probe
agaccaggtctctattttgaggaagaaataccgagacattgagcgactttgaggaatccg

SEQ ID NO: 215 - DNA, 60 bp, Artificial Sequence
  GPR180 probe
aagtcatgacacagtattcgctctttttctgaatgtttacatagagattcatcactgcag

SEQ ID NO: 216 - DNA, 60 bp, Artificial Sequence
  LPCAT1 probe
cagtaagtacgggaaaaaatgtttactaacttcctcagagattcgtgatacgcgtttctc

SEQ ID NO: 217 - DNA, 60 bp, Artificial Sequence
  SERF1A probe
ctttgaatggacataaaaattctgcttgttaagaacaagttgagctctggtaactgatct

SEQ ID NO: 218 - DNA, 60 bp, Artificial Sequence
  CDC42BPA probe
tgactgatgtgtctgaaaatgctaaggatcttattcgaaggctcatttgtagcagagaac

SEQ ID NO: 219 - DNA, 60 bp, Artificial Sequence
  NDC80 probe
ctctgaaagaagaagttcaaaagctggatgatctttaccaacaaaaaattaaggaagcag

SEQ ID NO: 220 - DNA, 60 bp, Artificial Sequence
  GMPS probe
atctgtggttattcgaacctttattactagtgacttcatgactggtatacctgcaacacc

SEQ ID NO: 221 - DNA, 60 bp, Artificial Sequence
  IGFBP5 probe
tccaccccaggacgccactcatttcatctcatttaagggaaaaatatatatctatctatt

SEQ ID NO: 222 - DNA, 60 bp, Artificial Sequence
  MMP9 probe
ggccctctcttctcacctttgttttttgttggagtgtttctaataaacttggattctcta

SEQ ID NO: 223 - DNA, 60 bp, Artificial Sequence
  CMC2 probe
ctgggttgatacctgaaagaatcctgtcttatttggtctccataatcctttgaatggaaa

SEQ ID NO: 224 - DNA, 60 bp, Artificial Sequence
  DIAPH3 probe
agtaccctgatatactgaattttgtggatgatttggaacctttagacaaagctagtaaag

SEQ ID NO: 225 - DNA, 60 bp, Artificial Sequence
  DIAPH3 probe
aagactttcttactgacctgaataacttcagaaccacattcatgcaagcaataaaggaga

SEQ ID NO: 226 - DNA, 60 bp, Artificial Sequence
  QSOX2 probe
tttagtggtccgttgcctctgaagatgtaaacaaacaaatacactatttctgggaacatt

SEQ ID NO: 227 - DNA, 60 bp, Artificial Sequence
  TMEM65 probe
atagaatatgtatatgtattctttgtctaccaactaccaaagaaacaaatactcctcagt

SEQ ID NO: 228 - DNA, 60 bp, Artificial Sequence
  NUSAP1 probe
acattgcttacttaaaagctacatagccctatcgaaatgcgaggattaatgctttaatgc

SEQ ID NO: 229 - DNA, 60 bp, Artificial Sequence
  DIAPH3 probe
accataaggcaattgagcacataacgaaaaatgatgcaataagaatgtatgcactctctt

SEQ ID NO: 230 - DNA, 60 bp, Artificial Sequence
  MIR210HG probe
cagcctttcctcatgtcaacacagttcacaatatagttttcaaagtacagtttaaaactc

SEQ ID NO: 231 - DNA, 60 bp, Artificial Sequence
  TSPYL5 probe
cctccccaaaataattagtaactggttgttctacttggtaatttgacaccctgttaataa